(19) United States
(12) Patent Application Publication
(10) Pub. No.: US 2022/0233238 A1
(43) Pub. Date: Jul. 28, 2022

(54) DELIVERY CATHETER AND METHOD OF DISEASE TREATMENT

(71) Applicant: Neurotronic, Inc., Plymouth, MN (US)

(72) Inventors: Lixiao Wang, Henderson, NV (US); John J. Chen, Plymouth, MN (US); The Thomas Trinh Tran, Coon Rapids, MN (US)

(21) Appl. No.: 17/610,472

(22) PCT Filed: Jun. 25, 2020
(86) PCT No.: PCT/US20/39605

§ 371 Date: Nov. 11, 2021

Related U.S. Application Data
(63) Continuation-in-part of application No. 16/690,992, filed on Nov. 21, 2019.
Application No. 16/690,992 is a continuation-in-part of application No. 16/563,235, filed on Sep. 6, 2019.
(60) Provisional application No. 62/866,266, filed on Jun. 25, 2019.

(51) Int. Cl. A61B 18/14; A61M 25/00; A61M 25/10

(52) U.S. Cl. CPC A61B 18/1492; A61B 2018/00285; A61M 25/1011; A61M 25/0084

(57) ABSTRACT

Embodiments of the present invention provide a device and a method for at least one disease by delivering an effective amount of energy and/or formulation to tissue on or near to a body lumen wall. The formulation can include at least one of a gas, a vapor, a liquid, a solution, an emulsion, a suspension of one or more ingredients, or a combination thereof. The amounts of the formulation and/or energy delivered can be effective to injure or damage tissue, nerves, and/or nerve endings to relieve disease symptoms.

### CROSS-REFERENCE TO RELATED APPLICATION

This application claims the benefit of priority to U.S. Provisional Patent Application Ser. No. 62/866,266 filed Jun. 25, 2019, U.S. patent application Ser. No. 16/563,235 filed Sep. 6, 2019, and U.S. patent application Ser. No. 16/690,992 filed Nov. 21, 2019, the disclosures of which are incorporated herein in their entirety by reference.

### BACKGROUND

Hypertension, or high blood pressure, is a major global health concern. An estimated 30 to 40% of the adult population in the world suffers from this condition. Furthermore, its prevalence is expected to increase, especially in developing countries. Diagnosis and treatment of hypertension remain suboptimal, and most patients struggle to properly control blood pressure.

Benign prostatic hyperplasia is a non-cancerous enlargement of the prostate gland, which affects more than 50% percent of men over the age of 60. Early in life, the prostate is approximately the size of a walnut, weighing about 20 grams. Prostate enlargement over time is thought to be normal. With age, the prostate gradually increases to at least twice its original size. Prostate growth causes pressure to build against the neighboring urethra, leading to narrowing of this latter organ and ultimately resulting in urinary obstruction which makes urinating difficult.

Chronic obstructive pulmonary disease (COPD) is associated with two major airflow obstruction disorders: chronic bronchitis and emphysema. Chronic bronchitis results from inflammation of the bronchial airways. The bronchial airways connect the trachea to the lungs. Emphysema is a disease, which results from over-inflation of alveoli, or the air sacs in the lungs. This condition causes shortness of breath. Approximately 16 million Americans suffer from COPD, the majority of which (80-90%) are lifetime smokers. COPD is a leading cause of death in the United. States.

Asthma is a chronic respiratory disease characterized by excessive narrowing of the airways and caused by inflammation of the airways, excess mucus production and airway hyper responsiveness. This narrowing of the airways makes breathing difficult and can significantly impact patients' lives, limiting participation in numerous activities. In severe cases, asthma attacks can be life-threatening. To date, there is no known cure for asthma.

Chronic sinusitis (CS) results from inflammation of the membrane lining in one or more paranasal sinuses and is typically associated with significant tissue damage. Approximately 37 million cases of CS are reported annually to the Centers for Disease Control and Prevention (CDC).

Diabetes is a metabolic condition, or combination of conditions, where an individual experiences high concentrations of blood glucose. The condition is caused either by insufficient production of insulin within the body or by failure of cells to respond properly to insulin. Glycated hemoglobin (HbA1c) is a marker of plasma glucose concentration and is clinically used for the diagnosis of diabetes. In humans, normal HbA1c levels are typically <6.0%, prediabetes HbA1c levels range from 6.0 to 6.4%, and diabetes HbA1c levels exceed 6.5%.

Diabetes is one of the leading causes of death and disability in the United States and in other developed countries. It is associated with long-term complications that affect almost every part of the body. It has been linked, for instance, to blindness, heart and blood vessel disease, stroke, kidney failure, amputations, and nerve damage.

Within the United States, diabetes affects approximately 8 percent of the population and has resulted in costs that approach $250 billion.

Diabetes is typically classified as either type 1 (also referred to as insulin-dependent diabetes or juvenile diabetes), wherein the patient fails to produce sufficient insulin, type 2 (also referred to as non-insulin-dependent diabetes, adult-onset diabetes, or obesity-related diabetes), wherein the patient fails to respond properly to insulin, or gestational diabetes, a condition which develops late in pregnant women.

Type 2 diabetes is the most common form of diabetes, accounting for 90 to 95% of overall cases. It is generally associated with older age, obesity, family history, previous history with gestational diabetes, and physical inactivity. It is also more prevalent in certain ethnicities. Type 2 diabetes is also referred to as insulin-resistant diabetes, as the pancreas typically produces sufficient amounts of insulin, but the body fails to respond properly it. Symptoms associated with type 2 diabetes include fatigue, frequent urination, increased thirst and hunger, weight loss, blurred vision, and slow healing of wounds or sores.

For the maintenance of normal glucose homeostasis, the liver is crucial, which produces glucose during fasting and stores glucose postprandially. However, these hepatic processes are dysregulated in type 1 and type 2 diabetes mellitus, and this imbalance contributes to hyperglycemia in the fasted and postprandial states. Net hepatic glucose production is the summation of glucose fluxes from gluconeogenesis, glycogenolysis, glycogen synthesis, glycolysis and other pathways. Glucose level is sensed by neurons and glial cells expressing glucose transporters (GLUT) in the central nervous system (CNS) and by peripheral tissues such as the taste buds, gut, and carotid body. In the liver, glucose level is also sensed at the portal veins. Activation of the sympathetic efferent nerves increases glucose production and suppresses glycogenesis.

To measure fasting blood glucose, a blood sample can be taken after an overnight fast. A fasting blood glucose level less than 100 mg/dL (5.6 mmol/L) is normal. A fasting blood glucose level from 100 to 125 mg/dL (5.6 to 6.9 mmol/L) is considered prediabetes. A measurement of 126 mg/dL (7 mmol/L) or higher on two separate tests is considered to indicate diabetes. The fast blood glucose levels in diabetes patients is in the range of 126 mg/dL to 400 mg/dL or even higher, or in the range of 126 mg/dL to 300 mg/dL, or in range of 126 mg/dL to 250 mg/dL. The liver acts as the body's glucose (or fuel) reservoir and helps to keep circulating blood glucose levels and other body fuels steady and constant. The liver both stores and manufactures glucose depending upon the body's need. The need to store or release glucose is primarily signaled by insulin and glucagon. During a meal, the liver stores sugar, or glucose, as glycogen for a later time when the body needs it. The high levels of insulin and suppressed levels of glucagon during a meal promote the storage of glucose as glycogen. When not eating—especially overnight or between meals—the body has to make its own sugar. The liver supplies sugar or glucose by turning glycogen into glucose in a process called glycogenolysis. The liver also can manufacture necessary sugar or glucose by harvesting amino acids, waste products and fat byproducts. This process is called gluconeogenesis. When the body's glycogen storage is running low, the body starts to conserve the sugar supplies for the organs that always require sugar. These include: the brain, red blood cells and parts of the kidney. To supplement the limited sugar supply, the liver makes alternative fuels from fats called ketones. This process is called ketogenesis. The hormone signal for ketogenesis to begin is a low level of insulin. Ketones are burned as fuel by muscle and other body organ, while sugar is saved for the organs that need it.

Glucagon, epinephrine, norepinephrine, cortisol, and growth hormone help maintain blood sugar levels and make the blood glucose rise. The concentrations of glucagon, epinephrine, norepinephrine, cortisol, and growth hormone are high in diabetes patients. Glucagon, made by islet cells (alpha cells) in the pancreas, controls the production of glucose and another fuel, ketones, in the liver. Glucagon is released overnight and between meals and is important in maintaining the body's glucose and fuel balance. It signals the liver to break down its starch or glycogen stores and helps to form new glucose units and ketone units from other substances. It also promotes the breakdown of fat in fat cells. Epinephrine and norepinephrine are very similar. Both are neurotransmitters, and both increase blood pressure and blood glucose levels. Epinephrine (adrenaline) is released from nerve endings and the adrenals and acts directly on the liver to promote glucose production (via glycogenolysis). Epinephrine also promotes the breakdown and release of fat nutrients that travel to the liver and that are converted into glucose and ketones. Cortisol is a steroid hormone also secreted from the adrenal gland. It makes fat and muscle cells resistant to the action of insulin and enhances the production of glucose by the liver. Under normal circumstances, cortisol counterbalances the action of insulin. Under stress or if a synthetic cortisol is given as a medication (such as with prednisone therapy or cortisone injection), cortisol levels become elevated and insulin resistance results. When a patient has Type 2 diabetes, this means they may need to take more medication or insulin to keep blood glucose under control. Growth hormone is released from the pituitary, which is a part of the brain. Like cortisol, growth hormone counterbalances the effect of insulin on muscle and fat cells. High levels of growth hormone cause resistance to the action of insulin.

Obesity is another significant health concern, particularly in the developed world. It is a complex, multifactorial and chronic condition characterized by excess body fat, which results from an imbalance between energy expenditure and caloric intake. Although the causes of this imbalance are not completely understood, genetic and/or acquired physiologic events and environmental factors are thought to contribute. The adverse health effects associated with obesity, and more specifically morbid obesity, have become well-established in recent years. Such adverse effects include, but are not limited to, cardiovascular disease, diabetes, high blood pressure, arthritis, and sleep apnea. Generally, as a patient's body mass index (BMI) rises, the likelihood of suffering the adverse effects linked to obesity also rises.

Metabolic syndrome is a serious health condition that affects up to one-third of U.S. adults and places them at higher risk of cardiovascular disease, type 2 diabetes, stroke, and diseases related to fatty buildups in artery walls. It is a cluster of conditions that occur together. These conditions include increased blood pressure, high blood sugar, excess body fat around the waist, and abnormal cholesterol or triglyceride levels. The National Institutes of Health guidelines define metabolic syndrome as having three or more of the following traits, including traits you are taking medication to control: large waist (a waistline that measures at least 35 inches (89 centimeters) for women and 40 inches (102 centimeters) for men), high triglyceride level (150 milligrams per deciliter (mg/dL), or 1.7 millimoles per liter (mmol/L), or higher of this type of fat found in blood), reduced “good” or HDL cholesterol (less than 40 mg/dL (1.04 mmol/L) in men or less than 50 mg/dL (1.3 mmol/L) in women of high-density lipoprotein (HDL) cholesterol), increased blood pressure (130/85 millimeters of mercury (mm Hg) or higher), elevated fasting blood sugar (100 mg/dL (5.6 mmol/L) or higher)

Nonalcoholic fatty liver is another health concern, which occurs when the liver has trouble breaking down fats, causing fat to build up in the liver tissue of people who drink little or no alcohol. It is normal for the liver to contain some fat. However, if more than 5% to 10% of the liver's weight is fat, then it is called a fatty liver (steatosis). Nonalcoholic fatty liver disease (NAFLD) is common and, for most people, causes no signs and symptoms and no complications. But in some people with nonalcoholic fatty liver disease, the fat that accumulates can cause inflammation and scarring in the liver. This more serious form of nonalcoholic fatty liver disease is sometimes called nonalcoholic steatohepatitis (NASH). NASH causes the liver to swell and become damaged. At its most severe, nonalcoholic fatty liver disease can progress to liver failure. NASH is associated with dyslipidemia, with low high-density lipoprotein (HDL) (<40 mg/dL in men or <50 mg/dL in women), hypertriglyceridemia (≥150 mg/dL), hypercholesterolemia (≥200 mg/dL), and triglycerides (TG)/HDL>5.0. NASH resolution is associated with decreases in TG and TG/HDL ratio.

The spleen is the main filter for blood-borne pathogens, as well as key organ for iron metabolism and erythrocyte homeostasis. The spleen also plays an important role in the immune response to inflammatory conditions including rheumatoid arthritis (RA), cancer, myocardial infarction, and atherosclerosis. The autonomic nervous system regulates of immunity, or responds to inflammatory stimuli by upregulating sympathetic nerve trafficking to the spleen, which can mobilize monocytes to sites of tissue injury and release cytokines that modulate the inflammatory response.

### SUMMARY OF THE INVENTION

Embodiments of the present invention relate to a delivery catheter for delivering chemical formulations and/or energy, and methods of treatment of at least one disease, such as by treating at least two different target tissues in one procedure. The at least one disease can include hypertension, pulmonary hypertension, diabetes, obesity, metabolic syndrome, heart failure, myocardial infarction, atherosclerosis, coronary artery disease (CAD), peripheral vascular disease (PAD), end-stage renal disease, digestive disease, nonalcoholic fatty liver disease (NAFLD), nonalcoholic steatohepatitis (NASH), urological disease, cancers, tumors, pain, rheumatoid arthritis (RA), asthma, chronic obstructive pulmonary disease (COPD), or a combination thereof. The delivery catheters can deliver an effective amount of energy or/and chemical formulation to target tissue in body to relieve the symptoms of the disease, such as reducing blood pressure for hypertension, reducing blood glucose level and A1C for diabetes, reducing body weight for obesity, reducing restenosis for coronary and peripheral diseases, reducing liver fat for nonalcoholic fatty liver disease (NAFLD) and nonalcoholic steatohepatitis (NASH), and reducing pain for cancers and arthritis. The energy can be radiofrequency, cryoablation, microwave, laser, ultrasound, high-intensity focused ultrasound energies, or a combination thereof. The target tissue can include renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, or a distal end of a right main renal artery branch), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, and urological lumens. The delivery catheter can include a radiofrequency catheter, a cryoablation catheter, a microwave catheter, a laser catheter, an ultrasound catheter, a high-intensity focused ultrasound catheter, or a combination thereof. The delivery catheter can include a combination of a balloon and infusion catheter, as well as other delivery devices. The formulations delivered by the delivery catheter can include gases, vapors, liquids, solutions, emulsions, suspensions of one or more ingredients, and combinations thereof. Delivery of the formulations to targeted tissues in the human body by chemical infusion from the delivery catheter can improve treatment safety and efficacy.

Various embodiments of the present invention provide a method for treating at least one disease including treating at least two different target tissues in at least two different body lumens. The method includes performing a treatment procedure on a body lumen that is a first body lumen. The treatment procedure includes inserting the delivery catheter into the body lumen. The delivery catheter includes a catheter shaft, a balloon at a distal end of the shaft, and an inflation lumen in fluid communication with an interior of the balloon. The treatment procedure includes inflating the balloon to center the distal end of the shaft in the body lumen. The treatment procedure includes denervating or ablating target tissue of the body lumen with the delivery catheter including delivering an amount of energy or formulation to the target tissue effective to injure or damage the target tissue to relieve a disease symptom. The treatment procedure includes deflating the balloon. The treatment procedure also includes removing the delivery catheter from the body lumen. The method also includes performing the treatment procedure on a second body lumen different than the first body lumen.

The method for treating at least one disease including treating at least two different target tissues in at least two different body lumens using the deliver catheter can be a method of treating metabolic syndrome. The method can include treating a first, second, third, and fourth target tissue, that are all different from one another, and are respectively located in a first, second, third, and fourth body lumen. The first body lumen can include a renal artery (e.g., left main renal artery, right main renal artery, a renal artery branch, a left renal artery branch, a right renal artery branch, a distal end of the left main renal artery, a distal end of the right main renal artery, a distal end of a left main renal artery branch, or a distal end of a right main renal artery branch), a renal vein, a pulmonary artery, a vascular lumen, a celiac artery, a common hepatic artery, a proper hepatic artery, a gastroduodenal artery, a right hepatic artery, a left hepatic artery, a splenic artery, a right gastric artery, a left gastric artery, a right suprarenal artery, a left suprarenal artery, a right inferior phrenic artery, a left inferior phrenic artery, a nonvascular lumen, an esophagus, a digestive lumen, the stomach, the duodenum, the jejunum, or a combination thereof. The second body lumen can include a right main renal artery, a left main renal artery, a right renal branch artery, a left renal branch artery, a common hepatic artery, a proper hepatic artery, a gastroduodenal artery, a right hepatic artery, a left hepatic artery, a splenic artery, a right gastric artery, a left gastric artery, a right suprarenal artery, a left suprarenal artery, a right inferior phrenic artery, a left inferior phrenic artery, or a combination thereof. The third body lumen can include a splenic artery, a gastric artery, a left gastric artery, or a combination thereof. The fourth body lumen can include a gastroduodenal artery, a gastric artery, a left gastric artery, a right suprarenal artery, a left suprarenal artery, a right inferior phrenic artery, a left inferior phrenic artery, or a combination thereof. The chemical formulation and/or energy delivered to the target tissues can reduce body weight, reduce high blood pressure, reduce A1C, reduce blood glucose level, reduce body waist fat tissue, or a combination thereof.

The method for treating at least one disease including treating at least two different target tissues in at least two different body lumens using the deliver catheter can be a method of treating hypertension. The method can include treating a first, second, third, and fourth target tissue, that are all different from one another, and are respectively located in a first, second, third, and fourth body lumen. The first body lumen can include a renal artery, such as a left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof. The second body lumen can include a common hepatic artery, a proper hepatic artery, a gastroduodenal artery, a right hepatic artery, a left hepatic artery, a splenic artery, a right gastric artery, a left gastric artery, or a combination thereof. The third body lumen can include a splenic artery, a right gastric artery, a left gastric artery, or a combination thereof. The fourth target tissue can include a gastroduodenal artery, a right gastric artery, a left gastric artery, a right suprarenal artery, a left suprarenal artery, a right inferior phrenic artery, a left inferior phrenic artery, or a combination thereof. The chemical formulation and/or energy delivered to the target tissues can reduce blood pressure.

The method for treating at least one disease including treating at least two different target tissues in at least two different body lumens using the deliver catheter can be a method of treating diabetes. The method can include treating a first, second, third, and fourth target tissue, that are all different from one another, and are respectively located in a first, second, third, and fourth body lumen. The first body lumen can include a common hepatic artery, a proper hepatic artery, a right hepatic artery, a left hepatic artery, or a combination thereof. The second body lumen can include a renal artery (e.g., left main renal artery, right main renal artery, a renal artery branch, a left renal artery branch, a right renal artery branch, a distal end of the left main renal artery, a distal end of the right main renal artery, a distal end of a left main renal artery branch, or a distal end of a right main renal artery branch), a splenic artery, a right gastric artery, a left gastric artery, or a combination thereof. The third body lumen can include a splenic artery, a right gastric artery, a left gastric artery, or a combination thereof. The fourth body lumen can include a gastroduodenal artery, a right gastric artery, a left gastric artery, a right suprarenal artery, a left suprarenal artery, a right inferior phrenic artery, a left inferior phrenic artery, or a combination thereof. The chemical formulation and/or energy delivered to the target tissues can reduce blood glucose level, reduce A1C, or a combination thereof.

The method for treating at least one disease including treating at least two different target tissues in at least two different body lumens using the deliver catheter can be a method of treating obesity. The method can include treating a first, second, third, and fourth target tissue, that are all different from one another, and are respectively located in a first, second, third, and fourth body lumen. The first body lumen can include a common hepatic artery, a proper hepatic artery, a right hepatic artery, a left hepatic artery, or a combination thereof. The second body lumen can include a renal artery (e.g., left main renal artery, right main renal artery, a renal artery branch, a left renal artery branch, a right renal artery branch, a distal end of the left main renal artery, a distal end of the right main renal artery, a distal end of a left main renal artery branch, or a distal end of a right main renal artery branch), a splenic artery, a right gastric artery, a left gastric artery, or a combination thereof. The third body lumen can include a splenic artery, a right gastric artery, a left gastric artery, or a combination thereof. The fourth body lumen can include a gastroduodenal artery, a right gastric artery, a left gastric artery, a right suprarenal artery, a left suprarenal artery, a right inferior phrenic artery, a left inferior phrenic artery, or a combination thereof. The chemical formulation and/or energy delivered to the target tissues can reduce body weight, reduce body mass index, or a combination thereof.

The method for treating at least one disease including treating at least two different target tissues in at least two different body lumens using the deliver catheter can be a method of treating nonalcoholic fatty liver disease (NAFLD), nonalcoholic steatohepatitis (NASH), or a combination thereof. The method can include treating a first, second, third, and fourth target tissue, that are all different from one another, and are respectively located in a first, second, third, and fourth body lumen. The first body lumen can include a common hepatic artery, a proper hepatic artery, a right hepatic artery, a left hepatic artery, or a combination thereof. The second body lumen can include a renal artery (e.g., left main renal artery, right main renal artery, a renal artery branch, a left renal artery branch, a right renal artery branch, a distal end of the left main renal artery, a distal end of the right main renal artery, a distal end of a left main renal artery branch, or a distal end of a right main renal artery branch), a splenic artery, a right gastric artery, a left gastric artery, or a combination thereof. The third body lumen can include a splenic artery, a right gastric artery, a left gastric artery, or a combination thereof. The fourth body lumen can include a gastroduodenal artery, a right gastric artery, a left gastric artery, a right suprarenal artery, a left suprarenal artery, a right inferior phrenic artery, a left inferior phrenic artery, or a combination thereof. The chemical formulation and/or energy delivered to the target tissues can reduce fat in the liver.

In various embodiments, the present invention provides a method for treating a disease. The method includes inserting a delivery catheter into a body lumen, wherein the delivery catheter includes a catheter shaft, at least one spray hole, and at least one marker band. The method includes spraying a formulation through the at least one spray hole, wherein the amount of the formulation delivered is effective to injure or damage target tissue to relieve a disease symptom. The method includes optionally removing the formulation from the tissue. The method also includes withdrawing the delivery catheter from the body lumen.

In various embodiments, the present invention provides a method for treating a disease. The method includes inserting a centering balloon delivery catheter into a body lumen, wherein the balloon delivery catheter includes at least one centering balloon and a catheter shaft, at least one injection needle, and at least one marker band. The method includes inflating the centering balloon to center the delivery catheter shaft in body lumen. The method includes deploying the at least one needle into a wall, outside the wall, or inside the wall of the body lumen. The method includes infusing a formulation through the at least one needle, wherein the amount of the formulation delivered is effective to injure or damage target tissue to relieve a disease symptom. The method optionally includes removing the formulation from the tissue. The method includes retracting the needles into the delivery catheter and deflating the centering balloon. The method includes withdrawing the delivery catheter from the body lumen.

In various embodiments, the present invention provides a centering balloon catheter for delivery of materials to a target location in the body lumen of a patient. The centering balloon catheter includes a proximal end; a distal end; a wire lumen; a balloon inflation lumen; a formulation infusion lumen and/or a vacuum lumen; an expandable balloon section; at least one injection needle; at least one marker band adjacent to the centering balloon; and at least one needle-exit opening adjacent to the marker band for deployment of the needle.

In various embodiments, the present invention provides a needle-based balloon delivery catheter for delivery of materials to a target tissue in the body lumen of a patient. The delivery catheter includes a catheter shaft with a proximal and distal end. The delivery catheter includes at least one marker band located near the distal end of the shaft. The delivery catheter includes at least one needle situated in a needle lumen, wherein the needle lumen is open to the outside of the catheter shaft via at least one needle exit hole. The delivery catheter includes a flushing port at the proximal end of the shaft in fluid communication with a flushing lumen, the flushing lumen being in fluid communication with a distal end of the needle lumen, wherein the flushing port is in fluid communication with the needle exit hole through the flushing lumen. The delivery catheter includes a guide wire lumen which extends through at least the distal end of the shaft. The delivery catheter includes at least one balloon adjacent to the distal end of the catheter. The delivery catheter includes an inflation lumen. The delivery catheter includes an inflation port in fluid communication with the inflation lumen and in fluid communication with an interior of the balloon. The balloon is inflatable via the inflation port through the inflation lumen and approximately centers the distal end of the catheter shaft in a body lumen. The delivery catheter includes an ablation or denervation port at the proximal end of the shaft. The ablation or denervation port is in fluid communication with the at least one needle for supplying ablation energy or formulation to the at least one needle. The delivery catheter also includes a needle movement controller in electrical or mechanical communication with the at least one needle. The needle movement controller deploys the at least one needle into the body lumen, into a wall of the body lumen, or outside of the body lumen.

In various embodiments, the present invention provides a delivery catheter. The delivery catheter includes a shaft with a proximal and distal end. The delivery catheter includes one or more needles for infusion treatment arranged near the distal end of the shaft. The delivery catheter includes an inflatable balloon arranged near the distal end of the shaft such that when the delivery catheter is placed in a lumen and the balloon is inflated, the distal end of the catheter shaft is centered in the lumen. The delivery catheter also includes a marker band at the distal end of the shaft.

In various embodiments, the present invention provides a delivery catheter. The delivery catheter includes a shaft with a proximal and distal end. The delivery catheter includes one or more needles for infusion treatment arranged near the distal end of the shaft. The delivery catheter also includes a steering mechanism associated with the shaft such that the distal end of the shaft is steerable in a direction away from the longitudinal axis of the shaft.

In various embodiments, the present invention provides a delivery catheter. The delivery catheter includes a shaft with a proximal and distal end. The delivery catheter also includes one or more needles for infusion treatment arranged near the distal end of the shaft. The delivery catheter further includes an inflatable balloon arranged near the distal end of the shaft such that when the delivery catheter is placed in a lumen and the balloon is inflated, the distal end of the catheter shaft is centered in the lumen, wherein the distal end of the shaft includes a marker band; or a steering mechanism associated with the shaft such that the distal end of the shaft is steerable in a direction away from the longitudinal axis of the shaft; or a combination thereof.

Embodiments of the present invention are directed to the treatment of hypertension, diabetes, obesity, metabolic syndrome, heart failure, myocardial infarction, atherosclerosis, coronary artery disease (CAD), peripheral vascular disease (PAD), end-stage renal disease, a digestive disease, nonalcoholic fatty liver disease (NAFLD), nonalcoholic steatohepatitis (NASH), urological disease, cancers, tumors, pain, rheumatoid arthritis, asthma, and chronic obstructive pulmonary disease (COPD) by delivery of an effective amount of a formulation to target tissues. Such formulations include gases, vapors, liquids, solutions, emulsions, suspensions of one or more ingredients, or a combination thereof. Methods involve controlled delivery of the formulations to lumen surfaces and tissues within the human body, resulting in modifications to those areas. Such methods can lead to denervation of nerves and nerve endings in and adjacent to the body lumens. The methods can also include the beneficial severing of nerves and nerve endings to interrupt nerve communication. Temperature may enhance the safety and efficacy of the treatment formulations. The temperature of the formulation can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. In some embodiments, the formulation includes one of binary, ternary, or quaternary components, and may include more than four components. Methods of delivery include a less invasive, percutaneous approach and a non-invasive approach. Embodiments of the present invention provide a formulation and a delivery catheter that enhances absorption and penetration of the formulation into body tissues and lumen nerves and nerve endings.

In one embodiment, the formulation includes water, saline, hypertonic saline, phenols, methanol, ethanol, absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate, urea, lipiodol, surfactants, derivatives thereof, or combinations thereof.

In one embodiment, at least one ingredient of the formulation is a gas. The gas includes one of oxygen, nitrogen, helium, argon, air, carbon dioxide, nitric oxide, vapors of organic and inorganic compounds, water, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate, derivatives thereof, and combinations thereof.

In one embodiment, at least one ingredient of the formulation is a surfactant. The surfactant includes PEG laurate, Tween 20, Tween 40, Tween 60, Tween 80, PEG oleate, PEG stearate, PEG glyceryl laurate, PEG glyceryl oleate, PEG glyceryl stearate, polyglyceryl laurate, polyglyceryl oleate, polyglyceryl myristate, polyglyceryl palmitate, polyglyceryl-6 laurate, polyglyceryl-6 oleate, polyglyceryl-6 myristate, polyglyceryl-6 palmitate, polyglyceryl-10 laurate, polyglyceryl-10 oleate, polyglyceryl-10 myristate, polyglyceryl-10 palmitate, PEG sorbitan monolaurate, PEG sorbitan monolaurate, PEG sorbitan monooleate, PEG sorbitan stearate, PEG oleyl ether, PEG laurayl ether, organic acid, salts of any organic acid and organic amine, polyglycidol, glycerol, multiglycerols, galactitol, di(ethylene glycol), tri(ethylene glycol), tetra(ethylene glycol), penta(ethylene glycol), poly(ethylene glycol) oligomers, di(propylene glycol), tri(propylene glycol), tetra(propylene glycol), penta(propylene glycol), poly(propylene glycol) oligomers, a block copolymer of polyethylene glycol and polypropylene glycol, Pluronic, Pluronic 85, derivatives thereof, or combinations thereof.

In one embodiment, the formulation includes at least one of an oil, a fatty acid, and a lipid. In some embodiments, at least one of an oil, a fatty acid, and a lipid in the formulation is chosen from butanoic acid, hexanoic acid, octanoic acid, decanoic acid, dodecanoic acid, tetradecanoic acid, hexadecanoic acid, octadecanoic acid, octadecatrienoic acid, eicosanoic acid, eicosenoic acid, eicosatetraenoic acid, eicosapentaenoic acid, docosahexaenoic acid, tocotrienol, butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, natural or synthetic phospholipids, mono-, di-, or triacylglycerols, cardiolipin, phosphatidylglycerol, phosphatidic acid, phosphatidylcholine, alpha tocoferol, phosphatidylethanolamine, sphingomyelin, phosphatidylserine, phosphatidylinositol, dimyristoylphosphatidylcholine, dioleoylphosphatidylcholine, dipalmitoylphosphatidylcholine, distearoylphosphatidylcholine, phosphatidylethanolamines, phosphatidylglycerols, sphingolipids, prostaglandins, gangliosides, neobee, niosomes, and derivatives thereof.

In another embodiment, the formulation includes a therapeutic agent or drug for nerve denervation. The therapeutic agent includes at least one of sodium channel blockers, tetrodotoxins, saxitoxins, decarbamoyl saxitoxins, vanilloids, neosaxitoxins, lidocaines, conotoxins, cardiac glycosides, digoxins, glutamates, staurosporines, amlodipines, verapamils, cymarins, digitoxins, proscillaridins, quabains, veratridines, domoic acids, ethanols, oleandrins, carbamazepines, aflatoxins, guanethidines, and guanethidine sulfates. In another embodiment, the formulation includes a contrast agent for imaging nerve denervation. Such contrast agents include one of iodine, ethyl iodide, sodium iodide, lipiodol, nonoxynol iodine, iobitridol, iohexol, iomeprol, iopamidol, iopentol, iopromide, ioversol, ioxilan, iotrolan, iodixanol, ioxaglate, derivatives thereof, and combinations thereof.

In one embodiment, the formulation includes an azeotrope. An azeotrope is a mixture of two or more ingredients that cannot be altered by simple distillation. This happens because the vapor produced upon boiling has constituents proportional to those of the original mixture. Potential formulation azeotropes include ethanol/water, ethanol/water/contrast agent, ethanol/water/surfactant, ethanol/water/contrast agent/surfactant, propanol/water, iso-propanol/water, butanol/water, acetic acid/water, or combinations thereof.

In one embodiment, the formulation is in a gaseous or vapor state and includes one or more ingredients. The vapor or gas formulation can include oxygen, nitrogen, helium, argon, air, carbon dioxide, nitric oxide, water, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate, or combinations thereof. In one embodiment, the vapor formulation includes one of binary, ternary or quaternary components, and may include more than four components. The vapor formulation can include an azeotrope or a contrast agent, such as lipiodol or iodine, and may include a surfactant and/or a therapeutic agent. The temperature of the vapor formulation can be 0 to 140° C., preferably from 15 to 100° C., most preferably from 20 to 85° C., or 0° C. or less, or less than, equal to, or greater than 10° C., 20, 30, 40, 50, 60, 70, 80, 90, 100, 110, 120, 130, or 140° C. or more.

In one embodiment, the formulation is in a liquid state and includes one or more ingredients. The liquid formulation may include one of water, saline, hypertonic saline, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, lipiodol, methyl acetate, ethyl acetate, ethyl nitrate, isopropyl acetate, ethyl lactate, urea, surfactant, and others. The liquid formulation may include an azeotrope or contrast agent and may include a therapeutic agent. In one embodiment, the formulation may include one of binary, ternary, or quaternary components, and may also include more than four components. In some embodiments, the liquid formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The liquid formulation may include a solution, a suspension, an emulsion, or a combination thereof.

In one embodiment, the method for treatment of at least one disease includes inserting a delivery catheter percutaneously and/or trans-orally into the target tissues in the human body; using the catheter to infuse a therapeutic formulation into the tissues of the body, wherein the amount of the formulation delivered is effective to beneficially injure or damage the tissues; optionally removing the formulation; and withdrawing the delivery catheters from the body. The injury or damage to the tissues can relieve disease symptoms, such as by lowering blood pressure, reducing glucose level, reducing body weight, relieving shortness of breath, relieving heart conditions, relieving vascular conditions, relieving join pain, relieving stiffness, relieving swelling, or a combination thereof. The at least one disease for this treatment include one or more of hypertension, pulmonary hypertension, diabetes, obesity, metabolic syndrome, heart failure, myocardial infarction, atherosclerosis, coronary artery disease (CAD), peripheral vascular disease (PAD), end-stage renal disease, a digestive disease, nonalcoholic fatty liver disease (NAFLD), nonalcoholic steatohepatitis (NASH), urological disease, cancers, tumors, pain, rheumatoid arthritis (RA), asthma, and chronic obstructive pulmonary disease (COPD). It may be more effective to treat multiple related diseases in one procedure, for example, for a patient who has combinations of two or more of hypertension, obesity, and type 2 diabetes. The tissues that can be treated can include renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, or a distal end of a right main renal artery branch), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, and urological lumens. The digestive lumens can include the esophagus, the stomach, the duodenum, the jejunum, the small and large intestines, and the colon. The formulations can include gases, vapors, liquids, solutions, emulsions, suspensions of one or more ingredients, or combinations thereof. If the formulation includes vapors of one or more ingredients, the heat can be generated by condensation of the vapors into liquids in the tissue. If the formulation includes liquids or solutions, the cooling or heat can be generated from formulation temperatures that fall below or exceed body temperature. The liquid formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. In one embodiment, the formulation temperature may be equal that of room temperature. In one embodiment, the formulation temperature can be −40 to −20° C. In another embodiment, the formulation temperature can be 15 to 80° C. In one embodiment, the formulation temperature may be equal that of body temperature. In another embodiment, the formulation temperature can be 50 to 80° C. In another embodiment, the temperature of the treated tissue may be lower than the formulation temperature and higher than body temperature. The temperature of the treated tissue can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more. In one embodiment, the temperature of the treated tissue can be −40 to −20° C. In another embodiment, the temperature of the treated tissue can be 15 to 80° C. In one embodiment, the temperature of the treated tissue may be equal that of body temperature. In another embodiment, the temperature of the treated tissue can be 50 to 80° C. The delivery catheter applicable to such a treatment includes a needle or needle-based catheter under imaged guide. The imaged guide includes one of ultrasound, X-ray, CT scan, MRI, OCT or scopes. The delivery catheter can also be balloon-based. Such balloon-based catheters can have both balloon(s) and needle(s) in one catheter. The delivery catheter can be spray-based. The spray-based catheter is capable of producing very fine mist to large droplets. In some embodiments, a combination of a balloon and spray catheter can be used in the procedure (e.g., free of needles), a combination of a needle catheter and a spray catheter can be used, or a combination of a needle catheter, a spray catheter, and a balloon catheter can be used (e.g., including both spray- and needle-based administration of the formulation). In one embodiment, the method includes flushing from the catheter's distal tip to protect and to dilute migrated chemical, and to prevent the runaway chemical from entering the distal portion of untreated area; flushing from delivery catheter; flushing from endoscope; removing or withdrawing the formulations from body tissues and lumens following treatment; and flushing the target area post-treatment with saline. If needles are present, the method can further include deploying the needles, administering the formulation from the needles, and retracting the needles.

In one embodiment, the delivery catheter includes at least one needle that is used to deliver the formulation into the vessel wall, to the outside of the vessel, or combinations thereof. In some of the needle-catheter embodiments, at least one balloon is used to approximately center the distal end of the catheter in the lumen, or to approximately center the portion of the catheter from which the needles emerge in the lumen. When the distal end of the catheter is centered in the treatment lumen, the user has better control over the needles and the injection depth. In some of the needle-catheter embodiments, there are two centering balloons and the at least one needle is positioned between the two balloons. In some of the needle-catheter embodiments, there is only one balloon and the at least one needle can be positioned either proximal or distal to the balloon, but in either case is adjacent to the balloon to take advantage of the catheter shaft being in the center of the treatment lumen. In one embodiment of the needle-catheter, there is not a centering balloon.

In one embodiment, the delivery catheter includes a spray catheter. In this embodiment, the formulation is delivered through spray holes at or near the distal end of the catheter so that it is delivered at a mist to the walls of the treatment lumen. This embodiment may include a sleeve positioned over the spray holes to provide for more even distribution of the formulation to the walls of the treatment lumen. This embodiment may include a vacuum port for removing excess formulation from the treatment lumen.

### BRIEF DESCRIPTION OF THE FIGURES

The drawings illustrate generally, by way of example, but not by way of limitation, various embodiments of the present invention.

FIG. 1 is an exemplary embodiment of a perspective view of a double balloon delivery catheter according to the present invention.

FIG. 2 is an embodiment demonstrating formulation infusion to an airway using a spray catheter.

FIG. 3 is an embodiment demonstrating formulation infusion to a renal artery using a triple-needle balloon delivery catheter.

FIG. 4 is an embodiment of a partial cross-sectional view of a double balloon delivery catheter in a body lumen.

FIG. 5 is an embodiment of a partial cross-sectional view of a triple-needle double-balloon delivery catheter in a body lumen with needles deployed.

FIG. 6A is an exemplary embodiment of a perspective view of an over-the-wire (OTW) triple-needle balloon delivery catheter according to the present invention.

FIG. 6B is an exemplary embodiment of a perspective view of an OTW triple-needle balloon delivery catheter with balloon and needles deployed according to the present invention.

FIG. 7A is another exemplary embodiment of a perspective view of a rapid exchange triple-needle balloon delivery catheter before needle deployment according to the present invention.

FIG. 7B is an exemplary embodiment of a perspective view of a rapid exchange triple-needle balloon delivery catheter with flushing lumen after needle deployment according to the present invention.

FIG. 7C is a transverse cross section at section 7C-7C of the catheter as shown in FIG. 7B, in accordance with various embodiments.

FIG. 7D is a transverse cross section at section 7D-7D of the catheter as shown in FIG. 7B, in accordance with various embodiments.

FIG. 7E is a transverse cross section at section 7E-7E of the catheter as shown in FIG. 7B, in accordance with various embodiments.

FIG. 7F is an alternative transverse cross section at section 7F-7F of the catheter shown in FIG. 7B, in accordance with various embodiments.

FIG. 7G is a transverse cross section at section 7G-7G of the catheter as shown in FIG. 7B, in accordance with various embodiments.

FIG. 8A is an exemplary embodiment of a perspective view of a steerable catheter before needle deployment.

FIG. 8B is an exemplary embodiment of a perspective view of a steerable catheter after the needle deployment.

FIG. 9A is an exemplary embodiment of a perspective view of a steerable catheter before needle deployment.

FIG. 9B is an exemplary embodiment of a perspective view of a steerable catheter with needle deployment.

FIG. 9C is an embodiment of a partial cross-sectional view of a one directional steerable catheter in a body lumen with needle deployed.

FIG. 10 is a bar chart demonstrating norepinephrine (NE) reduction following renal denervation from ethanol- vs. control-treated groups, in accordance with various embodiments.

FIG. 11 is a histopathologic image demonstrating severed renal nerves necrosis (as shown by black arrows) following ethanol treatment, in accordance with various embodiments.

FIG. 12 is a bar chart demonstrating norepinephrine (NE) reduction following hepatic denervation from ethanol- vs. control-treated groups, in accordance with various embodiments.

FIG. 13 is an exemplary embodiment of a perspective view of a spray catheter according to the present invention.

FIG. 14 is an exemplary embodiment of a perspective view of a spray catheter with dual-suction feature according to the present invention.

FIG. 15A is an embodiment of a formulation infusion to the left gastric artery with a triple-needle balloon delivery catheter.

FIG. 15B is an embodiment of a formulation infusion to the hepatic arteries with a triple-needle balloon delivery catheter.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the term “polymer” refers to a molecule having at least one repeating unit and can include copolymers.

In various embodiments, the present invention provides a method for treating at least one disease (e.g., one disease, two diseases, at least two disease, three diseases, at least three diseases, four diseases, or at least four diseases). The method includes using a delivery catheter in a body lumen, such as to treat at least two different target tissues in at least two difference body lumens. The method includes performing a treatment procedure on a body lumen that is a first body lumen. The treatment procedure can include inserting the delivery catheter into the body lumen. The delivery catheter can include a catheter shaft, a balloon at a distal end of the shaft, and an inflation lumen in fluid communication with an interior of the balloon. The treatment procedure can include inflating the balloon to center the distal end of the shaft in the body lumen. The treatment procedure can include denervating or ablating target tissue of the body lumen with the delivery catheter including delivering an amount of energy or formulation to the target tissue effective to injure or damage the target tissue to relieve a disease symptom. The treatment procedure can include deflating the balloon. The treatment procedure can also include removing the delivery catheter from the body lumen. The method can include performing the treatment procedure on a second body lumen that is different than the first body lumen.

Performing the treatment procedure on the second body lumen can include using the same delivery catheter or a different delivery catheter. Performing the treatment procedure on the second body lumen can include reusing the same delivery catheter in the treatment procedure on the second body lumen as used in the treatment procedure on the first body lumen. Performing the treatment procedure on the second body lumen can include using a different delivery catheter in treatment procedure on the second body lumen than used in the treatment procedure on the first body lumen, the different delivery catheter including a catheter shaft, a balloon at a distal end of the shaft, and an inflation lumen in fluid communication with an interior of the balloon.

Treating at least two different target tissues in at least two different body lumens means treating at least two classes of body lumens chosen from renal arteries, renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, and urological lumens. Renal arteries can include left main renal artery, right main renal artery, a renal artery branch, a left renal artery branch, a right renal artery branch, a distal end of the left main renal artery, a distal end of the right main renal artery, a distal end of a left main renal artery branch, and a distal end of a right main renal artery branch. Gastric arteries can include left gastric artery, right gastric artery, a left gastric artery branch, and a right gastric artery branch. Hepatic arteries can include hepatic artery, common hepatic artery, proper hepatic artery, left hepatic artery, middle hepatic artery, and a hepatic artery branch. Splenic arteries can include main splenic artery and a splenic artery branch. Suprarenal arteries can include right suprarenal artery and left suprarenal artery. Phrenic arteries can include right inferior phrenic artery and left inferior phrenic artery. Mesenteric arteries can include superior mesenteric artery, inferior mesenteric artery, and a mesenteric artery branch. Urological lumens can include urethra and ureter. For example, treatment of the left gastric artery and a left gastric artery branch is considered treatment of one class of body lumen. Treatment of the left gastric artery and the main splenic artery is considered treatment of two different classes of body lumen. Treatment of at least three different target tissues in at least three different body lumens, or of at least four different target tissues in at least four different body lumens, is correspondingly defined as treatment of three or four different classes of body lumens, respectively.

The target tissue of the first body lumen and the second body lumen can be different and can be independently selected from target tissues of renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, or a distal end of a right main renal artery branch), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, and urological lumens. The treated disease can be chosen from hypertension, pulmonary hypertension, diabetes, obesity, metabolic syndrome, heart failure, myocardial infarction, atherosclerosis, coronary artery disease (CAD), peripheral vascular disease (PAD), end-stage renal disease, a digestive disease, nonalcoholic fatty liver disease (NAFLD), nonalcoholic steatohepatitis (NASH), urological disease, cancers, tumors, pain, rheumatoid arthritis (RA), asthma, chronic obstructive pulmonary disease (COPD), and a combination thereof.

The relieving of the disease symptom includes relieving a symptom of hypertension, diabetes, obesity, coronary disease, peripheral disease, nonalcoholic fatty liver disease (NAFLD), nonalcoholic steatohepatitis (NASH), cancer, arthritis, or a combination thereof. Relieving the disease symptom can include reducing blood pressure, reducing blood glucose level and A1C, reducing body weight, reducing restenosis, reducing liver fat, and reducing pain, or a combination thereof.

The at least one disease treated by the method via treatment of different target tissues in the first and second body lumens can include at least two diseases, such as both renal hypertension and diabetes (e.g., with treatment of both the renal artery and the hepatic artery); or both renal hypertension and obesity (e.g., with treatment of both the hepatic artery and splenic artery); or both diabetes and obesity (e.g., with treatment of the splenic artery, hepatic artery, and the left gastric artery); or a combination thereof. The first or second body lumen can include the splenic artery. The first or second body lumen can include a renal artery (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof). The first or second body lumen can include the splenic artery. The first or second body lumen can include the main renal artery branch, the extra-renal artery branch, or a combination thereof. The first or second body lumen can include the hepatic artery, the hepatic artery branch, the right hepatic artery, the left hepatic artery, the common hepatic artery, the proper hepatic artery, the hepatic celiac artery, or a combination thereof. The first or second body lumen can include a renal artery (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof), and the method can result in renal norepinephrine reduction of at least 40%, or at least 10%, 15, 20, 25, 30, 35, 40, 45, or at least 50%.

The delivery catheter can further include a guide wire lumen extending at least through the distal end of the shaft, wherein the method can further include advancing the delivery catheter over the guidewire. The delivery catheter can further include a marker band on the balloon or adjacent to the balloon, wherein the method can further include monitoring a position of the marker band under fluoroscopy.

The delivery catheter can be a chemical infusion delivery catheter. The delivery catheter can be an energy-delivery catheter. The delivery catheter can be a combination chemical infusion delivery catheter and energy-delivery catheter. For an energy-delivery catheter, denervating or ablating target tissue of the body lumen with the delivery catheter can include delivering an amount of energy (e.g., thermal energy) or to the target tissue from the delivery catheter using radiofrequency, cryoablation, microwave, laser, ultrasound, high-intensity focused ultrasound, vapor condensation of at least some of the formulation to a liquid, or a combination thereof. The energy delivery catheter can therefore be one a radiofrequency catheter, cryoablation catheter, microwave catheter, laser catheter, ultrasound catheter, high-intensity focused ultrasound catheter, an infusion catheter, or a combination thereof.

The delivery catheter can be a radiofrequency energy-delivery catheter, or a combination radio frequency energy-deliver catheter and chemical infusion catheter. In some embodiments, safety and efficacy of the radiofrequency energy delivery is improved by increasing the size of ablation resulting from the energy delivery, while minimizing risks for complications that can arise during the energy delivery. Examples of such complications include thrombus formation, steam pops, bubbling, charring on the target tissue, restenosis, fibrosis in the media and the adventitia, and others related to catheter manipulation (i.e., perforations). Spot thermal ablation (RF ablation) is not uniform and it does not reach to the nerves in adventitia. Partially spot RF ablation leads to low efficacy (low blood pressure drops). A radiofrequency energy delivery catheter can include one or more electrodes. In various embodiments, the delivery catheter includes one or more needles, and one or more of the needles' tips can act as electrodes (e.g., the needles can be insulated to only leave the portion that is used for the electrode exposed, such as a 1 mm to 5 mm length uninsulated section of the needle; in other embodiments, the needles are not insulated). Needles used for radiofrequency ablation can be the same as the needles described herein for chemical infusion, or they can be different. For example, radiofrequency energy delivery needles can have any suitable diameter, can be solid or hollow, and can be used to deliver radiofrequency energy to the body lumen, to the body lumen wall, or outside the body lumen wall. For radiofrequency delivery, a return electrode can be positioned on the catheter or a device such as a mat can be used. The delivery catheter can include one or more electrodes on a wire (e.g., a wire having spring or shape-memory properties to ensure good contact with the target tissue), one or more electrodes on a polymer shaft, one or more electrodes on the outer surface of the balloon, or a combination thereof. The delivery catheter can include more than one electrode to enable shorter treatment time. The methods can include cooling the electrodes, such as via passive cooling via blood flow and/or cooling the electrodes via active fluid cooling, such as internal active cooling (closed-loop) or external active fluid cooling (open-loop). The electrodes can be reduced in size to increase passive cooling via blood flow. Cooling the electrodes can increase energy delivery into the nerve tissue of the target tissue. Cooling a needle electrode can include flowing a coolant or a liquid that enhances RF delivery thought the needle before, during, or after radiofrequency delivery (e.g., a chemical formulation as described herein or a different liquid composition). In one embodiment of external cooling of electrodes (open-loop), the chemical formulation described herein can replace the active cooling fluids. The disclosed formulations can be used not only for cooling electrodes, but also used for chemical ablation of the target tissue. The formulations can in some embodiments diffuse and permeate into the nerve tissue uniformly, and they can ablate the nerves in adventitia uniformly in the body lumen. Therefore, the chemical ablation formulation can be delivered during, before, and/or after the energy ablations.

For delivery catheters that deliver microwave or ultrasound, the delivery catheter can include a centering mechanism (e.g., centering balloon) and a cooling system for the energy source. A microwave or ultrasound energy source can be located inside of the balloon with cooling on the balloon surface or inside the balloon. Microwave or ultrasound energy sources can be focused such than only a subset of the circumference of the body lumen is treated, or the energy source can deliver energy around the entire circumference of the body lumen. For delivery catheters that deliver laser energy, the deliver catheter can be configured such that the laser energy is emitted from the wall of the shaft. For delivery catheters that can perform cryoablation (i.e., delivery of negative energy), needles or spray holes can be used to deliver the a cryoablation medium. During cryoablation, a cryoablation medium can be delivered to the target tissue, such as via a needle. Once the substance exits the delivery catheter, a pressure drop, evaporation, or phase change of the cryoablation medium can provide the cryoablation treatment. If a liquid is used for cryoablation, the needle or spray hole can be sealed to prevent delivery until the delivery catheter is positioned, and the delivery catheter can include a gas return channel to exhaust the vapor from the cryoablation medium to outside of the body. For performing cryoablation via spray holes, multiple balloons can be used to create a treatment window that contains the cryoablation fluid during the treatment and prevents cryoablation of body lumen tissue outside of the treatment window.

For any type of energy delivery, the method of using the delivery catheter can including cooling the energy source via the flushing lumen to reduce the temperature of the energy source and to clean the lumen (e.g., to clean the artery and prevent blood clogging). In various embodiments, a needle can be used for cooling. Delivery catheters for energy delivery can include a guidewire lumen to ease insertion and placement of the delivery catheter.

In some embodiments, the delivery catheter is electrode free. The delivery catheter can be free of any sources of ablative energy, such as free of sources of radiofrequency, ultrasound, and microwave energy.

Embodiments of the present invention are directed to the treatment of at least one disease by delivery of an effective amount of formulation and/or energy to target tissues. The disease can include hypertension, pulmonary hypertension, diabetes, obesity, metabolic syndrome, heart failure, myocardial infarction, atherosclerosis, coronary artery disease (CAD), peripheral vascular disease (PAD), end-stage renal disease, a digestive disease, nonalcoholic fatty liver disease (NAFLD), nonalcoholic steatohepatitis (NASH), urological disease, cancers, tumors, pain, rheumatoid arthritis (RA), asthma, chronic obstructive pulmonary disease (COPD), or a combination thereof. The cancers include adrenal, bladder, cervical, colon, esophageal, gallbladder, kidney, liver, lung, ovarian, pancreatic, prostatic, rectal, stomach, uterine, or combinations thereof. The formulations can include gases, vapors, liquids, solutions, emulsions, suspensions of one or more ingredients, or combinations thereof. The methods involve delivery of the formulation and/or energy to lumen surfaces, tissues and nerves in the human body in order to modify such surfaces, tissues and nerves. The tissues include renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, urological lumens, or a combination thereof. The digestive lumens include the esophagus, the stomach, the duodenum, the jejunum, the small and large intestines, the colon, or combinations thereof. The temperature may enhance the safety and efficacy of treatment formulations. The temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue may be different from the formulation temperature. The temperature of the treated tissue can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more. The amount of formulation and energy delivered may be effective to beneficially injure, damage, or eliminate (e.g., kill) target tissues and thereby relive disease symptoms such as by lowering blood pressure, shrinking tumors, relieving pain, relieving symptoms of asthma or COPD, or a combination thereof. The energy or heat can enhance the injury/damage/elimination effect by accelerating the reaction rate between the formulation and tissues. Methods of delivery include delivery of the formulations to ablate nerves that surround the human body lumens. The method can include removing or withdrawing the formulations from the tissue or lumen after treatment. Without direct observation, nerve ablation can be identified based on the physiologic function that corresponds to the nerves, such as glucose and norepinephrine (NE) levels. Norepinephrine is the main neurotransmitter used by the sympathetic nervous system, which is connected to numerous organs, including, for example, the heart, lungs, liver, spleen, gallbladder, stomach, intestines, kidneys, urinary bladder, and many other organs. For example, in the liver, increasing the sympathetic effects of norepinephrine will increase the production of glucose, either by glycogenolysis after a meal or by gluconeogenesis when food has not recently been consumed. Accordingly, the correction of overactive hepatic sympathetic nerves will reduce NE content and will result in a lower glucose level. In the kidneys, release of renin and retention of sodium in the bloodstream, which increase blood pressure. Norepinephrine (NE) content in the tissues can be used as a biomarker to demonstrate or indicate the efficacy of the treatment. Compared to an untreated (control) subject, various embodiments of the method including treatment of overactive hepatic sympathetic nerves can result in a NE content reduction in percentage of from 20% to 99%, preferably, 50% to 98%, and most preferably, 75% to 97%.

In one embodiment, the formulation is a single chemical or one of binary, ternary, or quaternary components, and may also include more than four components. In one embodiment, the delivery system can be used with less invasive percutaneous approaches or non-invasive approaches. Embodiments of the present invention provide a formulation including one or more ingredients that performs surface modification of the body lumen with absorption and penetration into tissues and nerves and nerve endings of the body lumen.

In one embodiment, the formulation can include or consist of water, saline, hypertonic saline, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate, urea, lipiodol, surfactant, derivatives thereof, or combinations thereof.

In one embodiment, the ingredient of the formulation is at least one gas. The gas can be chosen from oxygen, nitrogen, helium, argon, air, carbon dioxide, nitric oxide, vapors of organic and inorganic compounds, water, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate, and mixtures thereof.

In one embodiment, the ingredient in the formulation is at least one surfactant. In some embodiments, the surfactant is chosen from PEG laurate, Tween 20, Tween 40, Tween 60, Tween 80, PEG oleate, PEG stearate, PEG glyceryl laurate, PEG glyceryl oleate, PEG glyceryl stearate, polyglyceryl laurate, polyglyceryl oleate, polyglyceryl myristate, polyglyceryl palmitate, polyglyceryl-6 laurate, polyglyceryl-6 oleate, polyglyceryl-6 myristate, polyglyceryl-6 palmitate, polyglyceryl-10 laurate, polyglyceryl-10 oleate, polyglyceryl-10 myristate, polyglyceryl-10 palmitate, PEG sorbitan monolaurate, PEG sorbitan monolaurate, PEG sorbitan monooleate, PEG sorbitan stearate, PEG oleyl ether, PEG laurayl ether, organic acid, salts of any organic acid and organic amine, polyglycidol, glycerol, multiglycerols, galactitol, di(ethylene glycol), tri(ethylene glycol), tetra(ethylene glycol), penta(ethylene glycol), poly(ethylene glycol) oligomers, di(propylene glycol), tri(propylene glycol), tetra(propylene glycol), penta(propylene glycol), poly(propylene glycol) oligomers, a block copolymer of polyethylene glycol and polypropylene glycol, Pluronic, Pluronic 85, derivatives thereof, and combinations thereof. In some embodiments, the content of the surfactant in the formulation can be 0.1 to 80% by weight, preferably from 0.5 to 50% by weight, most preferably from 1 to 15% by weight.

In one embodiment, the formulation includes at least one of an oil, a fatty acid, and a lipid. The oil, fatty acid, and lipid in the formulation can be chosen from butanoic acid, hexanoic acid, octanoic acid, decanoic acid, dodecanoic acid, tetradecanoic acid, hexadecanoic acid, octadecanoic acid, octadecatrienoic acid, eicosanoic acid, eicosenoic acid, eicosatetraenoic acid, eicosapentaenoic acid, docosahexaenoic acid, tocotrienol, butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, natural or synthetic phospholipids, mono-, di-, or triacylglycerols, cardiolipin, phosphatidylglycerol, phosphatidic acid, phosphatidylcholine, alpha tocoferol, phosphatidylethanolamine, sphingomyelin, phosphatidylserine, phosphatidylinositol, dimyristoylphosphatidylcholine, dioleoylphosphatidylcholine, dipalmitoylphosphatidylcholine, distearoylphosphatidylcholine, phosphatidylethanolamines, phosphatidylglycerols, sphingolipids, prostaglandins, gangliosides, neobee, niosomes, derivatives thereof, and combinations thereof.

In an embodiment, the formulation includes a therapeutic agent or drug for nerve denervation and surface modification. The therapeutic agent is at least one of sodium channel blockers, tetrodotoxins, saxitoxins, decarbamoyl saxitoxins, vanilloids, neosaxitoxins, lidocaines, conotoxins, cardiac glycosides, digoxins, glutamates, staurosporines, amlodipines, verapamils, cymarins, digitoxins, proscillaridins, quabains, veratridines, domoic acids, ethanols, oleandrins, carbamazepines, aflatoxins, guanethidines, or guanethidine sulfates. In another embodiment, the formulation includes a contrast agent for imaging nerve denervation. Such contrast agents include iodine, ethyl iodide, sodium iodide, lipiodol, nonoxynol iodine, iobitridol, iohexol, iomeprol, iopamidol, iopentol, iopromide, ioversol, ioxilan, iotrolan, iodixanol, ioxaglate, derivatives thereof, and combinations thereof. The content of the contrast agent in the formulation can be 2 to 25% by weight, preferably from 5 to 15% by weight.

In one embodiment, the formulation includes an azeotrope. An azeotrope is a mixture of two or more ingredients that cannot be altered by simple distillation. This happens because the vapor produced upon boiling has constituents proportional to those of the original mixture. The azeotropes of the formulations can be chosen from ethanol/water, ethanol/water/contrast agent, ethanol/water/surfactant, ethanol/water/contrast agent/surfactant, propanol/water, iso-propanol/water, butanol/water, and acetic acid/water.

In one embodiment, the formulation is in a gaseous or vapor state, including one or more ingredients. In one embodiment, the gas or vapor formulation includes oxygen, nitrogen, helium, argon, air, carbon dioxide, nitric oxide, vapors of organic and inorganic compounds, or a combination thereof. The vapors of the organic and inorganic compounds include one of water, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate and their mixtures.

In one embodiment, the vapor formulation includes at least one of a contrast agent, such as lipiodol or iodine, and an azeotrope, and may also include a surfactant and/or a therapeutic agent. In one embodiment, the vapor is one of binary, ternary, or quaternary components, and may also include more than four components. The vapor formulation temperature can be 0 to 140° C., 15 to 100° C., 30 to 80° C., or 0° C. or less, or less than, equal to, or greater than 10, 20, 30, 40, 50, 60, 70, 80, 90, 100, 110, 120, 130, or 140° C. or more.

In one embodiment, the formulation is in a liquid state, including one or more ingredients. The liquid formulation includes at least one of water, saline, hypertonic saline, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, lipiodol, methyl acetate, ethyl acetate, ethyl nitrate, isopropyl acetate, ethyl lactate, urea, surfactant, others, and combinations thereof. In one embodiment, the liquid formulation includes one of a contrast agent and an azeotrope, and may also include a therapeutic agent. In one embodiment, the liquid formulation is one of binary, ternary, or quaternary components, and may also include more than four components. In one embodiment, the liquid formulation includes a solution, an emulsion, or a suspension. The liquid formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. In one embodiment, the formulation temperature may be room temperature. In one embodiment, the formulation temperature can be −40 to −20° C. In another embodiment, the formulation temperature can be 15 to 80° C. In one embodiment, the formulation temperature may be equal to that of body temperature. In another embodiment, the formulation temperature can be 50 to 80° C.

In one embodiment, the method for treatment of at least one disease includes inserting a delivery catheter percutaneously or trans-orally into the body; using the catheter to infuse a formulation and/or deliver energy to target tissues or lumens in the body; optionally removing or withdrawing the formulation from the target tissue or body lumen; and withdrawing the delivery catheters from the body. The one or more diseases for treatment can include hypertension, pulmonary hypertension, diabetes, obesity, metabolic syndrome, heart failure, myocardial infarction, atherosclerosis, coronary artery disease (CAD), peripheral vascular disease (PAD), end-stage renal disease, a digestive disease, nonalcoholic fatty liver disease (NAFLD), nonalcoholic steatohepatitis (NASH), urological disease, cancers, tumors, pain, rheumatoid arthritis (RA), asthma, chronic obstructive pulmonary disease (COPD), or a combination thereof. The cancers can include adrenal, bladder, cervical, colon, esophageal, gallbladder, kidney, liver, lung, ovarian, pancreatic, prostatic, rectal, stomach, uterine, or combinations thereof. The body tissues include renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, urological lumens, or a combination thereof. The digestive lumen can include the esophagus, the stomach, the duodenum, the jejunum, the small and large intestines, the colon, or combinations thereof. The formulations can include gases, vapors, liquids, solutions, emulsions, suspensions of one or more ingredients, and combinations thereof. In embodiments where the formulation includes vapors of one or more ingredients, the heat can be generated by condensation of the vapors into liquids in the tissue. In embodiments where the formulation includes liquids or solutions, cooling or heat can be generated from formulation temperatures that fall below or exceed body temperatures. The liquid formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. In one embodiment, the temperature of the treated tissue may be different from the formulation temperature and lower or higher than that of body temperature. The temperature of the treated tissue can be 15 to 100° C., more preferably from 20 to 90° C., most preferably from 36 to 80° C. In another embodiment, the temperature of the treated tissue can be −40 to −20° C. In some embodiments, the delivery catheter is a needle or a needle-based catheter under imaged guide. The imaged guide can be ultrasound, X-ray, CT-scan, MM, OCT, a scope, or a combination thereof. The delivery catheter can be a balloon-based needle catheter. A balloon-based needle catheter can have a single or double balloon. The balloon-based catheters can have single, double or triple needles. The infusion can be from a needle catheter component and can be defined as a needle infusion method. The infusion volume can be 0.1 mL to 5 mL, preferably from 0.2 mL to 2.5 mL, most preferably from 0.3 mL to 1.5 mL. If the delivery catheter is a balloon and needle infusion combination device, balloon pressure may be maintained in the range of 0.1 to 3 atm, preferably of 0.1 to 2 atm, and most preferably of 0.3 to 1 atm during the infusion time period. Optionally, this low-pressure balloon inflation can be operated and maintained with a syringe, and the balloon size can be viewed with fluoroscopy in real-time. The formulation infusion temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. More detailed examples of the catheter, such as single-balloon needle and double-balloon needle delivery catheters, are shown in the following sections.

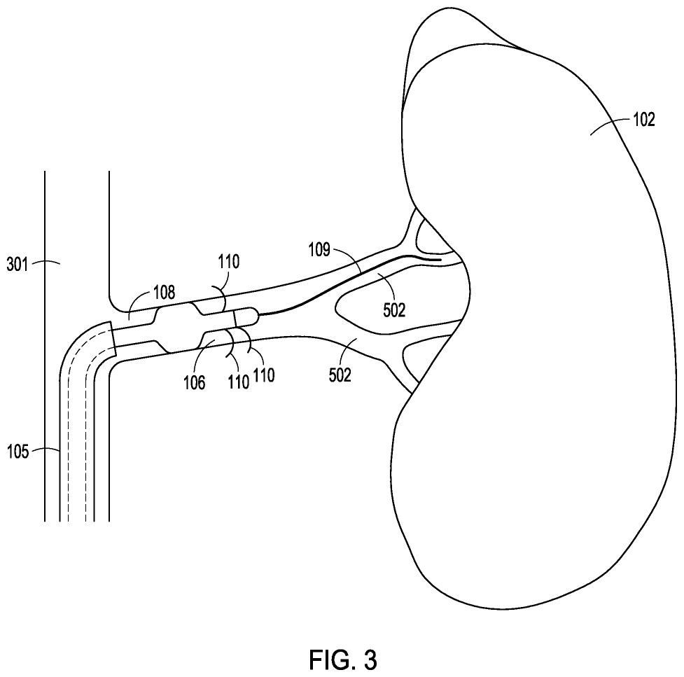

In one embodiment, the infusion lumen can be at least one needle, such as a single needle or as multiple needles (FIGS. 3 and 5-9). The needle tip(s) can be movable and, for delivery of the formulation, can be positioned in the wall of the lumen or outside of lumen wall by piercing through the lumen wall. The needles can be very small and can have a diameter (outside diameter, OD) of about 200 μm to 500 μm, preferably from about 300 μm to 400 μm. The small size of the needle can prevent or significantly reduce leakage or bleeding after the puncture and withdrawal. The needle devices can enable infusing the formulation into the adventitial layer of lumen tissues directly and precisely, and to achieve depth treatment. The infusion time of needle devices is within 3 minute, preferably between 5 seconds and 150 seconds.

In one embodiment, the formulation is ethanol or includes ethanol. The formulation can be delivered to the tissues of the body lumen as vapor or liquid. The vapor or liquid formulation temperature can be −40 to 150° C., preferably from −30 to 100° C., most preferably from −20 to 80° C. The temperature of the tissue can be −40 to 90° C., preferably from −30 to 80° C.

In one embodiment, the formulation is or includes a mixture of ethanol and water. The ethanol content can range from 10 to 100% by weight. This formulation can be delivered to the tissues of the body lumen as vapor or liquid. The vapor or liquid formulation temperature can be −40 to 150° C., preferably from −30 to 100° C., most preferably from −20 to 80° C. The temperature of the tissue can be −40 to 90° C., preferably from −30 to 80° C. The ethanol/water formulation can be a positive azeotrope. The azeotrope can be 95.63% ethanol and 4.37% water by weight. Ethanol boils at 78.4° C., water boils at 100° C., and the azeotrope boils at 78.2° C., which is lower than either of its constituents. 78.2° C. is the minimum temperature at which any ethanol/water solution can boil at atmospheric pressure.

In another embodiment, the formulation is a mixture of vapors including water, ethanol, and oxygen. In another embodiment, the formulation is a mixture of vapors including water, ethanol, and air. In another embodiment, the formulation is a mixture of vapors including water, ethanol, oxygen, and nitrogen. The formulations with oxygen and air can be especially useful for treating asthma and COPD.

In another embodiment, the formulation is a mixture of vapors including water, ethanol, and iodine, wherein an effective amount of the iodine vapor is included to image the mixture of vapors in the wall of the body lumen. In another embodiment, the formulation is a mixture of liquids including water and ethanol, and also including a surfactant. In another embodiment, the formulation is a mixture of liquids including water and ethanol, and also including a contrast agent, wherein an effective amount of the contrast agent can be included so as to be able to track the mixture in the wall of the body lumen by X-ray. The contrast agent can include iodine, ethyl iodide, sodium iodide, lipiodol, nonoxynol iodine, iobitridol, iohexol, iomeprol, iopamidol, iopentol, iopromide, ioversol, ioxilan, iotrolan, iodixanol, ioxaglate, derivatives thereof, and combinations thereof. The content of the contrast agent in the formulation can be 2 to 20% by weight, preferably from 5 to 15% by weight.

In one embodiment, the formulation is a mixture of acetic acid and water. The acetic acid content of the formulation can range from 1 to 100% by weight, preferably from 10 to 75% by weight, most preferably from 20 to 50% by weight. The formulation can be delivered to tissues of the body lumen as vapors or liquid. The vapor or liquid formulation temperature can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more. The temperature of the tissue can be −30 to 80° C., preferably from 60 to 80° C. or −30 to −20° C. The temperature of the tissue can be −40 to 0° C., preferably from −30 to −20° C. The acetic acid content in the formulation can be 2 to 75% by weight, preferably from 10 to 60% by weight.

In another embodiment, the formulation is a mixture of liquids including ethanol and lipiodol (e.g., LIPIODOL® ULTRA-FLUIDE), wherein an effective amount of lipiodol is included so as to be able to image the mixture of vapors in the wall of the body lumen and also to beneficially injure the target nerve tissue. The lipiodol content of the formulation can range from 10 to 80% by weight, preferably from 15 to 75% by weight, most preferably from 20 to 50% by weight. The formulation can be or include a mixture of liquids including water and lipiodol, or a mixture of liquids including acetic acid and lipiodol. The lipiodol content of the formulation can range from 10 to 80% by weight, preferably from 15 to 75% by weight, most preferably from 20-50% by weight.

In one embodiment, a delivery catheter is used to infuse the formulation to the tissues of the human body. The delivery catheter is a needle or needle-based balloon catheter and can be guided to the delivery site by X-ray or ultrasound-imaged guidance. The needle-based balloon delivery catheter can have one or two balloons. With a needle device, the formulation infusion can be in the lumen wall or outside the lumen wall (e.g., within or outside of the lumen).

In one embodiment, the delivery catheter is a spray catheter. The application of the formulation is can be to the inner wall of the body lumen. In some embodiments, the spray direction of the device is designed to be approximately perpendicular to the lumen wall. A spray catheter can be free of needles, or can include needles for both spray- and needle-based administration of the formulation. The spray catheter can include a centering balloon, or can be free of balloons. The spray catheter can optionally include the ability to delivery energy to the target tissue, such as radiofrequency, cryoablation, microwave, laser, ultrasound, high intensity focused ultrasound, or a combination thereof.

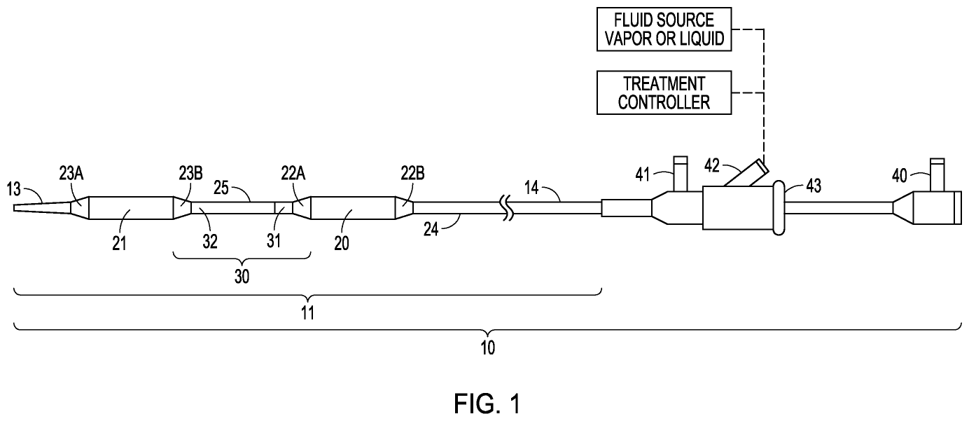

As shown in FIG. 1, a delivery catheter 10 has an elongated shaft 11 with at least one inner lumen, a distal end 13, and a proximal end 14. At the distal end 13 are proximal 20 and distal 21 lumen-conforming balloons. In any configuration, the tubing of the catheter shaft 11 may be extruded from plastic materials, e.g. thermoplastics, polyimides, polyetherimides, polyethylenes, polyurethanes, polyesters, polyamides, Pebax, nylons, fluorinated polyurethanes, polyether ether ketones, polysulfones, or the like, or combinations thereof. The catheter shaft 11 may be extruded or formed with a variety of lumen cross-sections, including circular or elliptic lumens. Further, as shown in FIG. 1, the catheter 10 may be equipped with a flushing port 43, a distal balloon inflation port 40 for inflation of the distal balloon 21 and a proximal balloon inflation port 41 for inflation of the proximal balloon 20, rendering the proximal 20 and distal 21 balloons separately inflatable. The lumen-conforming balloons are balloons that can be inflated at a pressure less than that needed to deform the lumen wall. The balloon material is selected to be flexible and usable at high temperatures, such that the balloon, when inflated, is compliant. In one embodiment, the balloon material is one of polyamides, nylons, Pebax, polyesters, polyethylene terephthalates or their copolymers. The inflated diameter of the balloons can range from about 2 millimeters to about 40 millimeters, depending on the diameter of the treatment site. In one embodiment, the diameter of each balloon is about 2 millimeters (“mm”). Alternatively, the inflated diameter of each balloon is less than, equal to, or greater than about 3 mm, 4, 5, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 mm or more.

In one embodiment, at least one marker band 22b is located proximally to the proximal balloon 20 and at least one marker band 23a is located distally to the distal balloon 21. The balloon catheter may be a rapid exchange or over-the-wire catheter made of any suitable biocompatible material. Marker bands can also be positioned on the other ends of balloons (22a and 23b). The segment 25 is in-between balloons 20 and 21 with at least one infusion hole, with non-expandable section 30 and the shaft proximal to the balloon section 24. Ports for balloon inflation 40 and 41 are for the distal and proximal balloons, respectively. Infusion port 42 is for infusion of the chemical formulation.

The material of balloon 20 and 21 includes polyesters, polyamides, nylon 12, nylon 11, polyamide 12, block copolymers of polyether and polyamide, Pebax, polyurethanes, block copolymers of polyether and polyester, or combinations thereof. The diameter of balloon 21 is equal to or less than that of balloon 20.

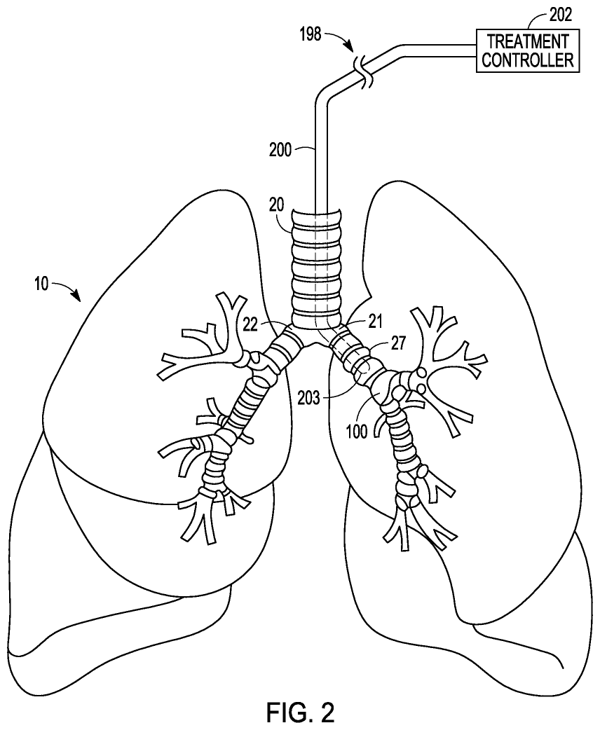

A schematic view of an embodiment of a spray catheter positioned within the left main bronchus for treatment of asthma and COPD is shown in FIG. 2. The delivery catheter 198 of FIG. 2 can treat airways that are distal to the main bronchi 21 and 22. For example, the delivery catheter 198 can be positioned up to 6^{th} or even 8^{th} generation airways to affect remote distal portions of the bronchial tree 27. The delivery system 198 can be navigated through tortuous airways to perform a wide range of procedures, such as, for example, denervation of a portion of a lobe, an entire lobe, multiple lobes, or one or both lungs. In some embodiments, the lobar bronchi are treated to denervate lung lobes. For example, one or more treatment sites along a lobar bronchus may be targeted to denervate an entire lobe connected to that lobar bronchus. Left lobar bronchi can be treated to affect the left superior lobe and/or the left inferior lobe. Right lobar bronchi can be treated to affect the right superior lobe, the right middle lobe, and/or the right inferior lobe. Lobes can be treated concurrently or sequentially. In some embodiments, a physician can treat a lobe. Based on the effectiveness of the treatment, the physician can concurrently or sequentially treat additional lobe(s). In this manner, different isolated regions of the bronchial tree can be treated.

The delivery catheter 198 can also be used in segmental or sub-segmental bronchi. Each segmental bronchus may be treated by delivering the formulation and/or energy to a single treatment site along the segmental bronchus. For example, the formulation and/or energy can be delivered to each segmental bronchus of the right lung. In some procedures, one or two applications of the formulation can treat most of or the entire right lung. Depending on the anatomical structure of the bronchial tree, segmental bronchi can often be denervated using one or two applications.

The delivery catheter 198 can affect nerve tissue while maintaining the function of other tissues or anatomical features, such as the mucous glands, cilia, smooth muscle, body lumens (e.g., blood vessels or other body lumens), and the like. Nerve tissue can include nerve cells, nerve fibers, dendrites, and supporting tissue, such as neuroglia. Nerve cells transmit electrical impulses, and nerve fibers are prolonged axons that conduct the impulses. The electrical impulses are converted to chemical signals to communicate with effector cells or other nerve cells. By way of example, the delivery catheter 198 is capable of denervating a portion of an airway of the bronchial tree 27 to attenuate one or more nervous system signals transmitted by nerve tissue. Denervating can include severing (by the treatment of the invention) the nerve tissue of a section of a nerve trunk to prevent signals from traveling through that specific area to more distal locations along the bronchial tree. If a plurality of nerve trunks extends along an airway, each nerve trunk can be severed. As such, the nerve supply along a section of the bronchial tree can be cut-off. When the signals are cut-off, the distal airway smooth muscle relaxes, leading to airway dilation. This airway dilation reduces airflow resistance so as to increase gas exchange in the lungs, thereby alleviating or eliminating one or more clinical manifestations, such as breathlessness, wheezing, chest tightness, and the like. Tissue surrounding or adjacent to the targeted nerve tissue may be affected but not permanently severed. In some embodiments, for example, the bronchial blood vessels along the treated airway can deliver a similar amount of blood to the bronchial wall tissues, and the pulmonary blood vessels along the treated airway can deliver a similar amount of blood to the alveolar sacs at the distal regions of the bronchial tree 27 before and after treatment. These blood vessels can continue to transport blood to maintain sufficient gas exchange. In some embodiments, airway smooth muscle is not beneficially injured to a significant extent. For example, a relatively small section of smooth muscle in an airway wall which does not appreciably impact respiratory function may be reversibly altered by the method, such as by employing the formulation at a regulated temperature to avoid irreversibly injuring nerve tissue outside of the airways such as the non-targeted smooth muscle tissue.

The delivery system 198 of FIG. 2 includes a treatment controller 202 and an intraluminal elongate assembly 200 connected to the controller 202. The elongate assembly 200 can be inserted into the trachea 20 and navigated into and through the bronchial tree 27 with or without utilizing a delivery assembly such as a guidewire. The elongate assembly 200 includes a distal tip 203.

The controller 202 of FIG. 2 can include one or more processors, microprocessors, digital signal processors (DSPs), field programmable gate arrays (FPGA), application-specific integrated circuits (ASICs), memory devices, buses, power sources, pumps, formulation resources, vapor resources, liquid resources, contrast resources, vapor generators, desired temperature formulation generators, and the like.

The distal tip 203 of FIG. 2 can target various sites in the lungs 10, including, without limitation, nerve tissue, fibrous tissue, diseased or abnormal tissue, muscle tissue, blood, blood vessels, various anatomical features (e.g., membranes, glands, cilia, and the like), or combinations thereof.

In one embodiment, a schematic view of a single balloon needle delivery catheter (a triple-needle balloon catheter is shown) positioned within a renal artery is shown in FIG. 3 with needles 110 shown extended. The delivery catheter 108 of FIG. 3 can treat hypertension. The formulation can be infused into the wall or the outside wall of the renal arteries adjacent to the renal nerves via needles for denervation. The nerve ends and small nerve fibers are typically located in the arterial wall, and the large nerve fibers and bundles usually located outside of or adjacent to the artery wall. Different levels of denervation can therefore be achieved by the control of needle penetration depth in the procedure. Some of the elements of the renal vascular system are omitted in FIG. 3. In FIG. 3, 102 is the kidney, 105 is the guiding catheter, 106 is main renal artery, 108 is a triple-needle balloon catheter, 109 is a guidewire, 301 is abdominal aorta, and 502 are extra-renal arteries. In FIGS. 3-5, 6A-6B, 7A-7G, 8A-8B, and 9A-9C, needle delivery catheters are shown and described herein with respect to delivery of chemical formulations via the needle; however, embodiments of the needle delivery catheter can additionally or alternatively be used for energy delivery to the target tissue in the form of radiofrequency, cryoablation, microwave, laser, and/or ultrasound, such as via the needle (e.g., radiofrequency, cryoablation), and/or via another source (e.g., microwave, laser, ultrasound).

In another embodiment, the delivery catheter 108 of FIG. 3 includes a balloon inflated to approximately center the catheter shaft, so the needles are equally spaced from the vessel wall and can advance through the artery wall at similar depths when they are deployed as shown. In some embodiments, the renal arteries are treated to treat hypertension. Because the needle penetrates the vessel wall or beyond the vessel wall, the device is capable of infusing the formulation into the vessel wall or to outside the outside wall. FIG. 3 shows the formulation is infused via needles 110 to the outer wall of the main renal arteries directly onto the renal nerves (not shown) for denervation.

In another embodiment, a triple-needle balloon catheter can be positioned within the extra renal arteries 502 and can infuse the formulation into the wall or outside the wall of these vessels. While three needles are shown, any suitable number of needles can be used, such as one, two, three, four, five, or more.

In another embodiment, one denervation procedure can treat both main and extra renal arteries. A triple-needle balloon catheter is positioned within main renal artery 106 and extra renal arteries 502 in FIG. 3 and infuses the formulation into the wall or outside the wall of the multiple renal arteries. Optionally, the arteries can be treated at multiple locations depending upon the artery length.

In one embodiment, the method for treatment of hypertension includes inserting a delivery catheter percutaneously into the renal artery and/or extra-renal arteries adjacent to nerves and nerve endings; using the delivery catheter to infuse the formulation described above and/or energy to the tissue of the body lumen adjacent to the nerves, wherein the amount of the formulation and/or energy delivered is effective to beneficially injure the nerves and nerve endings; and withdrawing the delivery catheter from the body lumen. The treatment will result in lower blood pressure for the treated patient.

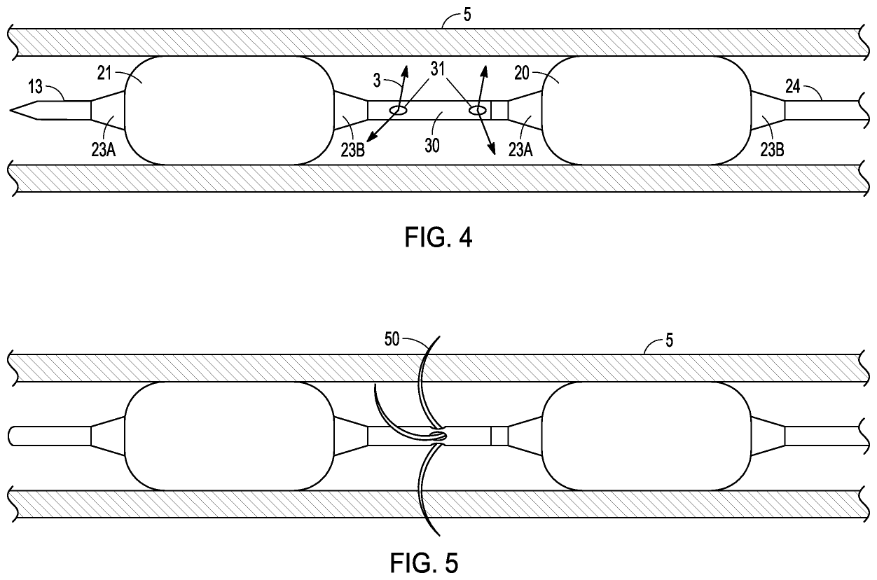

In one embodiment, a needle delivery catheter, for example, as shown in FIGS. 3 and 5-9, can be used for hypertension treatment via renal ablation, diabetes treatment via hepatic denervation, and/or obesity treatment via gastric denervation. In one embodiment, the catheter 10 (in FIG. 1) disclosed herein helps regulate formulation flow and treatment dose throughout the treatment window 30, as shown in FIG. 4. Balloons can be inflated through their inflation lumen. The position, diameter, number and frequency of needle-exit holes 31 results in the formulation being delivered to the treatment window 30 via needles (not shown, in retracted position). FIG. 4 depicts a catheter positioned in a body lumen 5 having two needle-exit holes 31 located within the treatment window 30 for delivery of the therapeutic agent via needles. During use, the needles extend outwardly from the needle-holes, as shown by 3. The needle-exit holes 31, as shown in FIG. 4, include retracted needles that are in fluid communication with the inner lumen 25. The needles that are within needle-exit holes 31 located within the treatment window 30 can be in communication with either the outer lumen 24 or inner lumen 25 (not shown in figure) such that the formulation is delivered to the treatment window 30 (e.g., delivered within the lumen, to the wall of the lumen, or outside the wall of the lumen).

Formulation treatment ports can be located between the balloons on the shaft. The formulation infusion holes can be located on the non-expandable shaft section between the expandable balloon sections. During treatment, the formulation can be discharged between the balloons through the infusion hole to the treatment window space created by the balloons. This is an infusion embodiment in which the treatment mechanism is to apply agent to the inside of vessel wall and can include allowing the agent to diffuse out through the lumen wall to achieve the therapeutic efficacy.

In one embodiment, optionally, the residual of the chemical agent/formulation may be retrieved by vacuum technique on one or more of the infusion holes following treatment. In this case, at least two treatment lumens are preferred: one for infusion and the other for vacuum. The formulation infusion and vacuum holes can be located on the non-expandable shaft section between the expandable balloon sections.

In addition to the withdrawal of excess treatment agent, left-over agent can also be diluted with saline or water to an ineffective concentration. Flushing with saline or water can be performed using a catheter wire lumen, one of the infusion lumens, or by other means. The method of use depends upon the site of protection or treatment. If the distal portion of vessels requires protection from the formulation treatment, flushing can be performed via wire lumen.

In one embodiment, when a deep treatment to the adventitial and/or peri-adventitial space is required, a needle-based catheter can achieve the goal as the needle-based catheter can infuse formulation into the lumen wall or to the outer wall of lumens through the at least one needle. In order to treat the lumen uniformly around its circumference, a three (or more) needle balloon catheter infuses formulation into target area via the needles. FIG. 5 depicts a double-balloon triple-needle catheter positioned in a body lumen 5, which has three needles, 50, deployed in-between the two balloons. When advanced, the three needles can have a larger diameter than the balloon diameter. The needles are in fluid communication with formulation source and deliver the formulation on to the vessel wall or into the outer wall of the body lumen directly. In a procedure, the balloon-needle catheter advances to targeted lesion or position per standard procedure, both balloons are inflated to center the middle non-expendable shaft, then needles are deployed from the middle non-expendable shaft and formulation is delivered via the needles to the wall or outer wall or outside the wall of the lumen or vessel depending upon the needle penetration depth, e.g., the needle tips can be in the wall or beyond the wall. After the treatment, the needles are retracted into the shaft and the balloons are deflated, and the catheter is ready to withdraw.

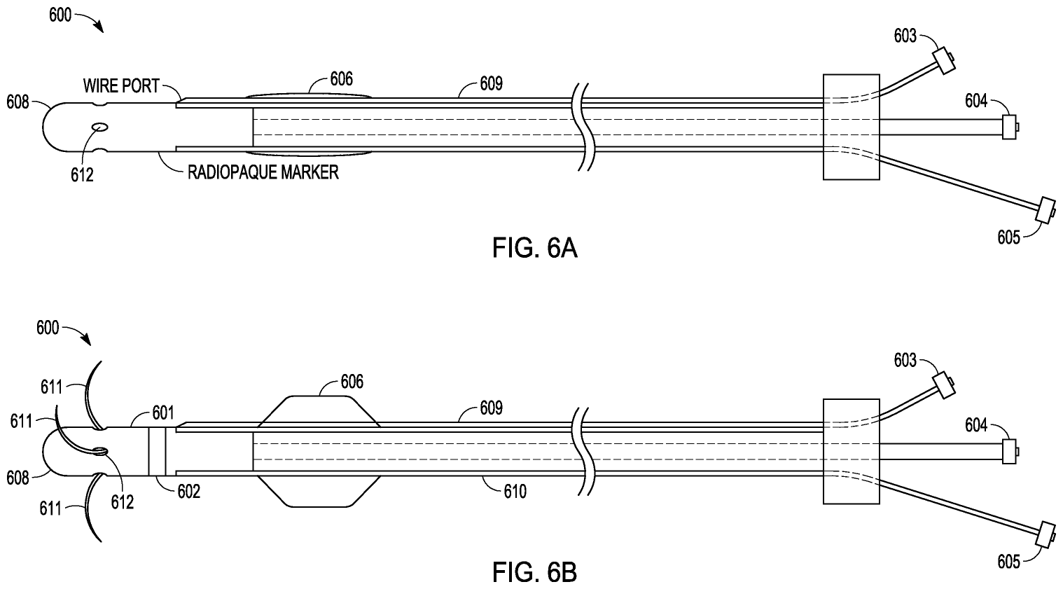

The examples of single-balloon needle catheter are shown in FIGS. 6A and 6B. FIG. 6A shows a triple-needle balloon catheter, 600, in a ready-to-use state. The needles are contained inside of the distal head 601 (not shown in figure) that is the distal part of catheter, in which the needle-containing voids (not shown) and corresponding needle-exit openings 612 are positioned distal of the balloon, with needle-exit openings allowing the needles to protrude from the voids at the time of needle deployment. The distal head can function as a marker band or, alternatively, can include marker band 602, and can be made from any medical grade metals, such as stainless steel, and can be atraumatic (tip 608) and radiopaque (e.g., when used as the marker band). The distal head and/or marker band can be visible under X-ray (fluoroscopy), which enables physician to position the needles accurately. The catheter has an off-center wire lumen design 603 which allows for smaller catheter profile, e.g., better compatibility with guide catheters. The catheter shaft 610 may be a reinforced shaft such as reinforced by wire braiding to enhance its compression resistance and tensile strength so the needle movement operated at the proximal end of catheter can be smooth and precise. The needle movement is controlled by a handle 604 located at the proximal end that is connected to a formulation source. The catheter, which can be advanced over a guide wire for delivery, is shown with an over-the-wire configuration for the guidewire lumen 603. The balloon, 606, can be inflated via inflation lumen 605. FIG. 6B shows the catheter 600 with the needles 611 in an advanced state with the balloon 606 inflated. The balloon, located just behind the needles, can center the distal end of shaft 610, so all three needles 611 can penetrate vessel wall at similar depths to ensure the formulation delivery evenly around the circumference of the vessel. It can be desirable to have the balloon positioned as close to the needle as possible, so that needles are more likely to be an equal distance from the vessel wall. The distance between needle-exit opening on the distal head and balloon cone/waist transition area can be from about 2 mm to 12 mm, preferably, from about 5 mm to 9 mm. A smaller needle diameter can result in a the less significant needle puncture wound, which can result in faster puncture wound healing. The needle outer diameter can be from about 0.20 mm to 0.50 mm; preferably, from about 0.28 mm to 0.38 mm. The needle curvature can be designed to enable needles 611 to align with vessel wall perpendicularly when they are delivered.

The needle can be formed of any suitable material, such as stainless steel, platinum, titanium, tantalum, platinum-iridium alloy, platinum-chromium alloy, nickel-titanium alloy (nitinol); a cobalt-based alloy including platinum (Pt), or gold (Au), or iridium (Ir), or osmium (Os), or rhenium (Re), or tungsten (W), or palladium (Pd), or tantalum (Ta), and combinations thereof, and/or chromium (Cr), and/or molybdenum (Mo) and/or nickel (Ni). The needle can be made of nitinol material and can have shape memory for the designed curvature upon exiting the containing holes/compartment. When the nitinol needle is small and thin, it may not have sufficient radiopacity under X-rays during the procedure. However, it is important for physician to know where the needles are during the procedure. To accomplish this, the needle can be modified with many suitable radiopaque metal materials by, for examples, thin film sputtering method on its surface with a radiopaque material(s), or the attachment of the radiopaque material(s) to the needle (e.g., as a marker band can be included on the shaft), or a clad metal needle/tube to combine a layer or two of radiopaque material(s) with the nitinol needle/tube. The coating or cladding or additional attachment material(s) can include, but not limited to, tungsten (W), Gold (Au), tantalum (Ta), platinum (Pt), and iridium (Ir), or combinations thereof, or their alloys, to render the nitinol needle radiopaque. The radius of curvature of needles 611 can be from about 2 mm to 6 mm, such as from about 3 mm to 4 mm. In a fully deployed state, needle tip-to-tip span diameter (the diameter of an imaginary circle that touches the tips of all the needles) can be 5 mm to 40 mm, or about 5 mm to 30 mm, such as about 5 mm to 15 mm; which can be dictated by treatment vessel diameter and desired needle penetration depth. For example, for a 6 mm vessel and 2 mm needle penetration depth, the needle tip-to-tip span distance can be about 10 mm.

After treatment, the needles are retracted back into the distal head or the containing compartment first, then balloon is deflated, and the catheter is withdrawn. Optionally, the distal head, needles and the treated vessel area can be flushed with saline or heparinized saline via wire lumen (for over-the-wire catheter) or a designated lumen to dilute residual formulation in the lumen if there is any. In some procedures, the formulation lumen 604 may be flushed with saline or heparinized saline to prevent needle clogging. While FIGS. 6A and 6B are shown with three needles located distal to the balloon, any number of needles such as one, two, three, four, five, or more, can be used and the needles can also be located proximal of the balloon. The needle guide can optionally be flushed.

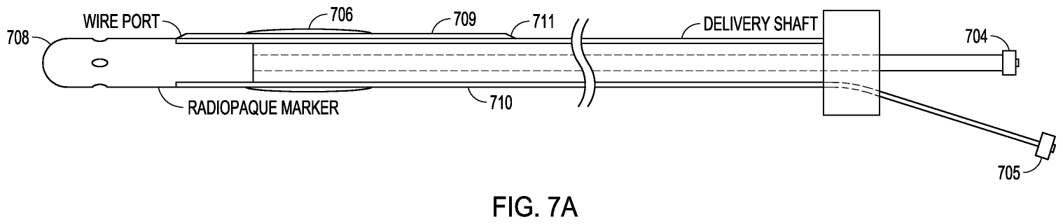

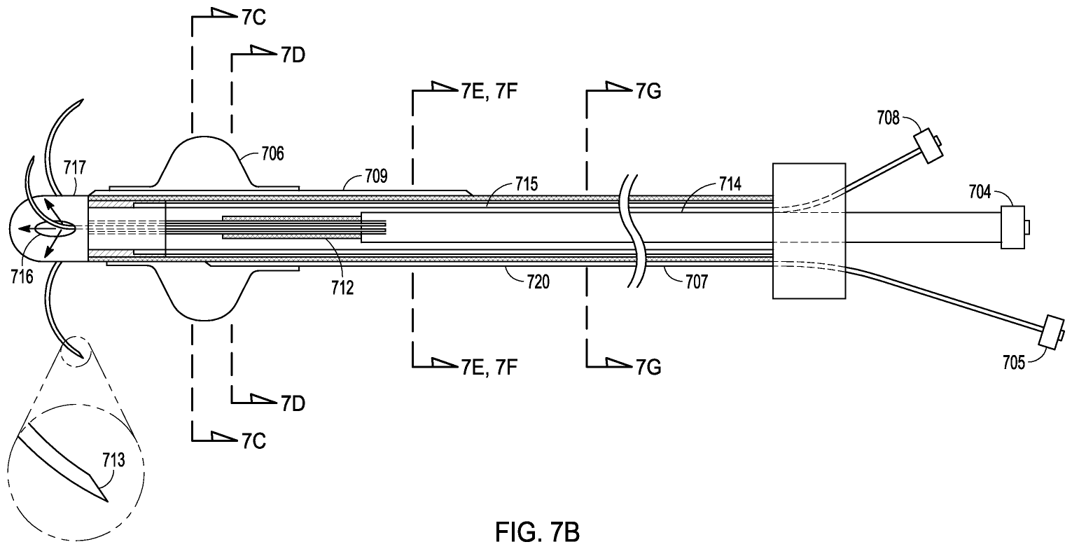

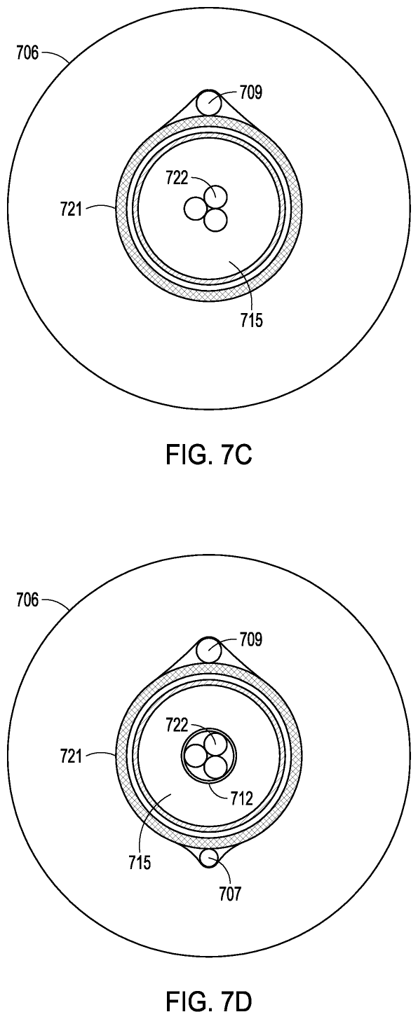

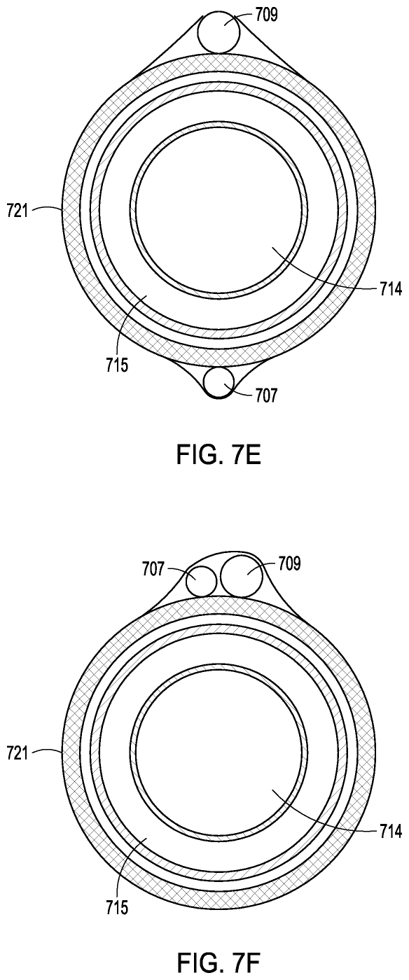

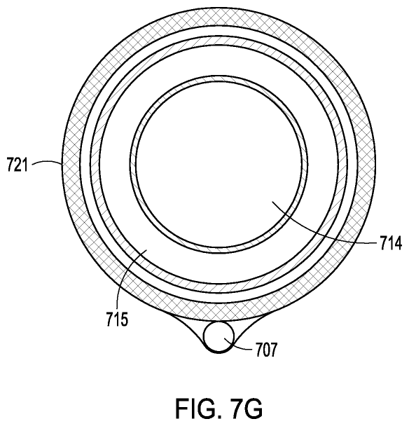

An embodiment of a rapid exchange (RX) needle balloon catheter is displayed in FIG. 7A, including a rapid exchange shaft. The rapid exchange configuration allows for easier and faster catheter insertion after the wire placement in the procedure. Guidewire lumen 709 has wire entry port or exit port 711 located near the middle or distal end of shaft 710. Optionally, there is a guiding groove on the distal head (not shown) to ease wire feeding for the physician during wire back feeding at the distal port of wire lumen. Except for the wire exchange method, the same needle and balloon operation procedure as described in the above paragraphs can be applied. Optionally, flushing and cleaning after the treatment for the distal head and needles can be done by injecting saline or heparinized saline directly via formulation lumen 704 at about 3 to 10 times of the treatment volume.

A needle-based balloon infusion system can include a needle-based balloon catheter, a guiding wire, an ablation agent, saline or heparinized saline or therapeutic medicine, and a set of fluid storages with volume of 1 mL to 30 mL for medical fluids management. Examples of fluid storages can include syringes or syringe pumps.

A needle-based balloon delivery catheter can include a distal head, at least one marker band, at least one needle, at least one needle exit hole, at least one flushing hole (which can, in some embodiments, be the same as the needle exit hole), a flushing lumen, a flushing port, a guide wire lumen, at least one balloon at an approximately distal end of the catheter, one inflating lumen, one inflating port, an ablating port, a needle movement shaft, and a needle movement controller; wherein the balloon is inflated via the inflating port through the inflating lumen; the flushing port is in communication to the flushing hole through the flushing lumen, the guide wire lumen is connected to the groove of the distal head; the needle movement controller is in communication with the needle through the needle movement shaft; the ablating port is in communication with the needle through the needle movement shaft; the needle is connected to the needle movement shaft; and needle movement is controlled by the needle movement controller through the needle movement controller. In one embodiment, the number of the needles is 1, 2, 3, 4, or 5, and the number of the needle exit holes is 1, 2, 3, 4, or 5. In another embodiment, the size of the catheter is 4F, 5F, 6F, 7F, 8F, 10F, 12F, 14F, 16F, 18F, 20F, 22F, 24F, or 26F. The balloon diameter can be in the range of 3 mm to 40 mm. The balloon length can be in the range of 3 mm to 25 mm. The needle size or needle outer diameter (OD) can be in the range of 0.3 mm to 0.5 mm, and the needle inside diameter (ID) can be in the range 0.2 mm to 0.35 mm. The needle span diameter when the catheter contains two needles or more can be in the range of 5 mm to 80 mm, or 5 mm to 40 mm, defined by their circumference of the needle tip points when the needles are fully advanced. The catheter length can be in the range of 70 cm to 260 cm. The guide wire lumen can be compatible with 0.014 inches (0.36 mm) or 0.018 inches (0.46 mm) or 0.035 inches (0.89 mm) guide wire. The needle span diameter can be fixed or adjustable; for example, the needle span diameter range can be adjustable from 5 mm to 80 mm, 5 mm to 30 mm, or 7 mm to 15 mm.

The needle can be to be visible under X-ray (fluoroscopy). The needle can be made of stainless steel, nitinol, cobalt-chromium, metal alloys, and shape memory metals and their alloys. The needle can be at straight or at curved shape. The metal needle radiopacity can be enhanced by the surface modification of another metal or two such as W, Au, Ta, Pt, Ir, and any combination thereof with physical vapor deposition (PVD) by sputtering of the another metal(s).

A needle-based balloon delivery catheter can include needle(s) movement control and ablation agent port. The ablation agent passes the port to the needle directly.

A needle-based balloon delivery catheter can include a wire lumen for guiding wire. The wire lumen can be featured as either an over-the-wire (OTW) version or rapid exchange (RX) version. When it is OTW shaft (catheter), a guidewire goes through the full length of the catheter shaft. As a counterpart, when it is RX shaft (catheter), a guidewire does not use the lumen through the catheter shaft. Typically, RX wire lumen is much shorter with the length of about 10 inches (about 25 cm), which is located at distal portion of the catheter. RX catheter saves time compared with advancing a guidewire through the full length, especially during the catheter exchange in the procedure.

A needle-based balloon delivery catheter can include at least one balloon. Short length balloon is preferred, and the balloon length can be in the range of 3 mm to 20 mm, preferably 5 mm to 10 mm long. Balloon diameter can be 3 mm to 30 mm. A required balloon compliance is largely dependent upon its application and can be in the range of compliant to noncompliant. The balloon material can be polyethylene, polyamide and its block copolymer, polyurethane, polyester and its block copolymer, nylon 12, Pebax, and so on. A standard balloon inflation medium can be used via inflation lumen/port to inflate and deflate the balloon. The inflation device can be a syringe or a balloon inflation device, however, the suitable volume syringe is preferred due to the nature of the balloon if the balloon is a compliant and low-pressure balloon.

The balloon on the catheter can be located behind the needle(s) to play the roles of centering the distal head of needle(s) and blocking the blood flow into the targeted treatment area.

A needle-based balloon delivery catheter can include a marker that indicates balloon location during the procedure. The marker can be a regular marker band used for a balloon catheter and can be a radiopaque metal (distal) head such as in the needle-based balloon catheter. The balloon marker can be placed at the distal, or the middle, or the proximal of the balloon, or the combination of the locations. Radiopaque marker bands can be made of platinum, platinum-iridium alloy, gold metal, and polymer composites with radiopaque fillers.

A needle-based balloon delivery catheter can include a flushing lumen that communicates directly with the needle-exit hole(s) for fluid exiting/passing. The flushing lumen can be connected via the flushing lumen port with the source of saline or heparinized saline for flushing and cleaning purpose, or of therapeutic medicines for inner surface treatment. The flushing lumen can be a co-axial structured (see in FIG. 7B) or with a separated tubing channel (not showing).

A needle-based balloon delivery catheter can include a balloon, three needles, a distal head of needle compartment and a radiopaque marker, a guide wire lumen, a balloon inflation lumen and port, an infusion lumen and port, and a flushing lumen and port.

An example of the rapid exchange (RX) needle balloon delivery catheter is illustrated in FIG. 7B. The catheter shaft 720 includes an independent flushing lumen 715. The flushing lumen 715 is connected to needle-exit holes 716 throughout the catheter shaft and to the flushing port 708, so the flushing medium exits thru the needle-exit holes as indicated with arrows. In this example, the flushing lumen is coaxially arranged over the infusion lumen 714. The infusion lumen 714 is directly connected to the needle tubes, which can be made of polymer tubes or metal tubes, preferably, a stainless steel hypotube. The three needles are bundled with a needle bundle tube 712. The needle bundle tube can be strong and flexible to hold the needles together and to keep the needles apart evenly, which can be a polymer tube or a reinforced polymer tube such as a braided polymer tube. The balloon 706 located behind the needle head plays a role of centering the needles in the vessel to ensure the three needles are at equal distance for the vessel wall. Needle distal head 717 is a needle holding compartment and a marker for needle placement in the procedure. The shape memory needle returns to the pre-shaped curvature upon exiting the distal head. It has a beveled needle distal opening 713 with the bevel facing the intrados direction of the needle. The needle distal opening communicates with the infusion lumen 714 and infusion port 704.

Various sections of the needle balloon delivery catheter shown in FIG. 7B are shown in FIG. 7C to FIG. 7G, which are the transverse cross-sectional views of the sections. FIG. 7C is a cross-sectional view of section 7C-7C of the catheter in FIG. 7B, which includes balloon body 706, a guide wire lumen 709, a braided outer shaft 721, three needle tubes 722 communicating with the distal head directly, and a flushing lumen 715.

FIG. 7D illustrates a cross-sectional view of section 7D-7D of the catheter in FIG. 7B, including balloon 706, a guide wire lumen 709, an inflation lumen 707, a flushing lumen 715, a braided outer shaft 721, needle tubes 722 with the needle bundle tubing (a braided tube) 712 to hold the needles together and to keep the needles apart evenly. As to the case of three needles, the needle-to-needle separation is 120° apart.

FIGS. 7E and 7F illustrates a cross-sectional view of sections 7E-7E and 7F-7F, respectively, of the catheter in FIG. 7B. FIG. 7E includes a guide wire lumen 709 and an inflation lumen 707, both of which are arranged on the top of the braided outer shaft but at opposite direction. FIG. 7E also includes a flushing lumen 715, a braided outer shaft, and an infusion lumen 714. The infusion lumen can be made of a polymer tube, or reinforced polymer tube, or a metal tube. For a better pushability and dimensional stability, a stainless steel (SS) hypotube can be used as the infusion lumen 714. This infusion lumen 714 of the catheter connects to the infusion port 704 at the proximal end and connects to the needle tubes at the distal shaft, of the catheter. Alternatively, to save the space or to lower the overall catheter profile, the guide wire lumen 709 and the inflation lumen 707 can be co-located at the same side of the shaft, as shown FIG. 7F. This configuration of FIG. 7F is not shown in the catheter of FIG. 7B.

FIG. 7G illustrates a cross-sectional view of section 7G-7G of the catheter in FIG. 7B, including a braided outer shaft with an inflation lumen 707 attached on its outer wall, a flushing lumen, and an infusion lumen of the SS hypotube. The inflation lumen 707 communicates with the inflation port 705 for balloon inflation. The infusion lumen 714 communicates with the infusion port 704 and the needle(s) and needle distal opening 713. The flushing lumen 715 communicates with the flushing port 708 and flushing hole(s) 716.

A rapid exchange (RX) needle balloon delivery catheter can be used in an ablation procedure via blood vessel. The infusion lumen and needles of the catheter can be prefilled with the ablation agent of ethanol from its port 704; and the flushing lumen can be prefilled with the medium such as saline or heparinized saline from its port 708. The prefilled flushing liquid in the distal head can reduce the probability of needle blockage because it prevents the needles from the direct contact of blood. Less blockage can mean a longer lasting catheter that can be used for multiple treatments in a procedure, and can help to shorten the procedure time and to lower the overall cost due to fewer catheters used. Then the prepared catheter can be inserted into the blood vessel within a guide catheter and along the pre-positioned guidewire to the treatment site. Once at the pre-determined treatment site, the balloon can be inflated to the vessel diameter first to center the needle head, then three needles can be advanced into the blood vessel wall or beyond, and ablation agent can be infused thru needles to the intended area for denervation. Alternatively, a therapeutic agent can be used instead of the ablation agent, for a therapeutic treatment other than ablation. After the treatment, the needles can be retracted back into the distal head, then flushing medium can be injected and flushed out thru needle-exit holes for flushing and cleaning, and then the balloon can be deflated, and the catheter can then be ready to withdraw or to move to next treatment site for another ablation. The flushing medium can also be a therapeutic medicine when a drug delivery is desired. The liquid drug can be applied via flushing holes to the site and its internal surface for treatment.

A flushing lumen on the catheter can be a safety mechanism for the patient. For example, it can dilute any excessive ablation solution or any residual of the ablative solution inside of vessel to the level of non-functioning.

The needle-based balloon delivery catheter can be a triple-needle balloon delivery catheter. The span diameter of the needles (calculated from the circumference formed by the needles tip-to-tip at a fully advanced stage) can be 5 mm to 30 mm, or 7 mm to 15 mm.

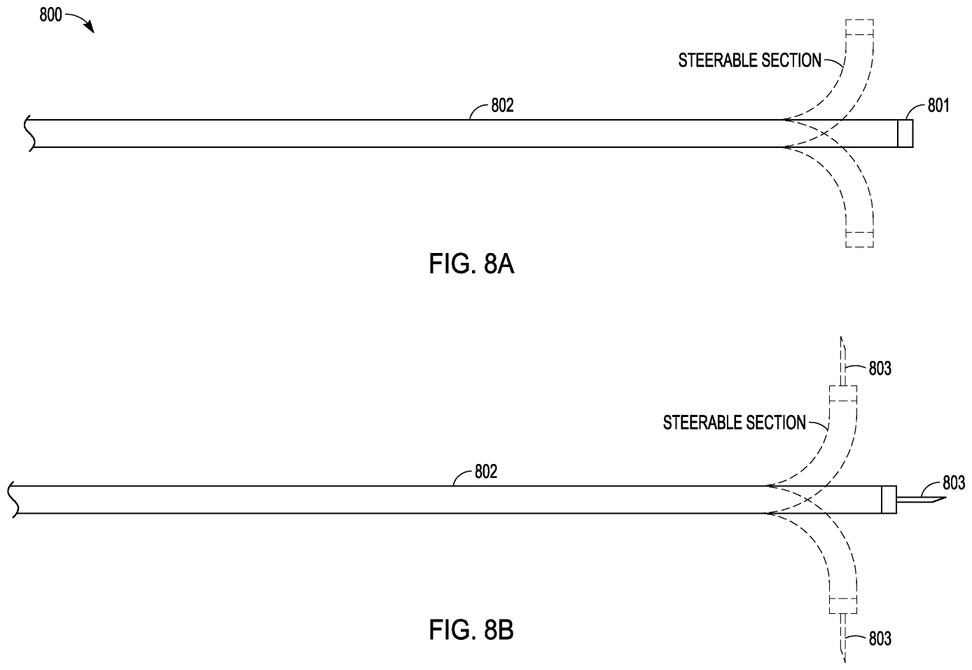

In one embodiment, shown in FIGS. 8A and 8B, a steerable single needle catheter can be used to infuse the formulation. As shown, the catheter has bidirectional steering, although one, two, three, four, or more directional capability can be used. The steering mechanism can be, for example, a wire (not shown) attached to the distal tip 801 of catheter shaft 802 with a handle (not shown) at the proximal end of the catheter. To steer the catheter, the user moves the handle in a proximal direction which causes the distal tip 801 to move away from the longitudinal axis of catheter shaft 802. The needle tip is retracted inside of catheter during insertion and positioning, which can be done with a guide wire. An off-center guide wire lumen design can be used to minimize catheter profile for small vessel access. The overall catheter profile is from 3F to 10F, preferably 5F to 7F. Once the treatment site is reached, the catheter head 801, which can be radiopaque, is steered towards and preferably against the vessel wall. This can be visualized guidance with fluoroscopy. Next, the needle (803 in FIG. 8B) is advanced into the vessel wall or through the wall and the formulation is infused. If desired, the needle can be retracted, and the tip can be steered to a new direction for treatment in the new direction. After the treatment, the needle is retracted, and then the catheter is withdrawn from the patient. Since the catheter is visible under X-ray and has a steerable tip, optionally, the steerable catheter can be used without a guide wire.

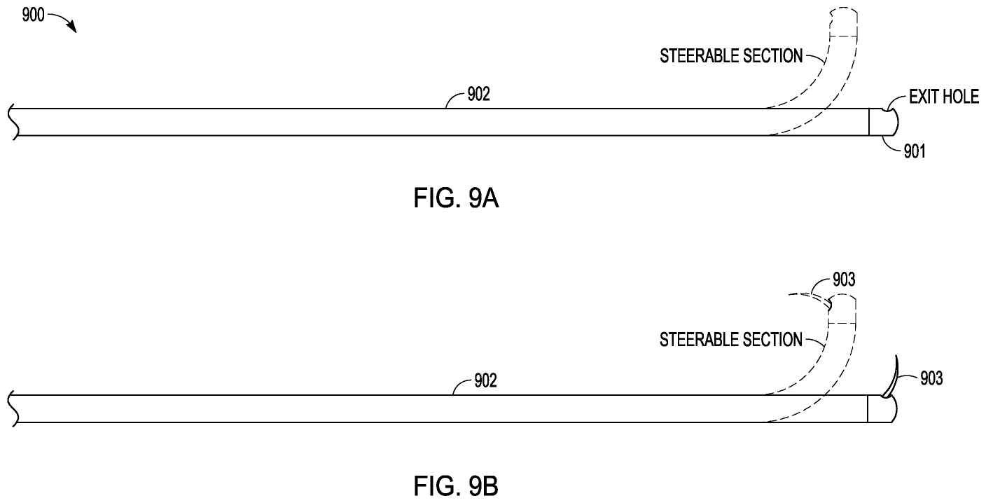

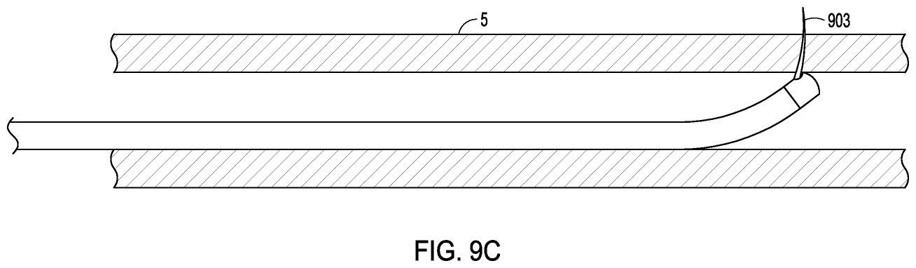

In another embodiment, shown in FIGS. 9A, 9B, and 9C, a single directional steerable catheter is used to infuse the formulation via the needle. This catheter 900 has smaller profile than the bi-directional catheter described above and is suitable for smaller diameter vessels. As described above, a desirable and controlled puncture can include the needle achieving an approximately perpendicular orientation to the lumen wall (e.g., 90° or close to 90° to the lumen wall), so the infusion direction and depth can be predicted. This may require a sharp bend at the distal end of catheter 902 so that tip 901 has a perpendicular alignment to the vessel wall. This can be very difficult in a small lumen as it can be almost impossible to push a needle through a small bend or curvature. To overcome this problem, in some embodiments the exit hole of the needle is placed at the side wall of the tip head instead of at the middle of the tip 903 (see FIGS. 9B and C). In addition, the exit hole is positioned on the side of catheter shaft 902 that is in the same direction as the catheter tip bending direction (see FIG. 9B). When the catheter is placed in a small lumen at the target area, it only needs to bend by a small angle (less than 90°) to align the hole in an approximately perpendicular fashion against the vessel wall 5 (see FIG. 9C). Once the alignment is achieved, the needle is advanced into the wall 5 as shown in FIG. 9C and the system is ready for infusion treatment. After the treatment, the needle is retracted into the catheter, the catheter tip returned to straight position and the catheter is ready to be withdraw from the patient, rotated for treatment is a different orientation, or moved to a different location for another treatment. The distal tip can be made of plastic, metal, or a combination thereof, which can be radiopaque and can contain the needle. The radiopaque tip 901 can act as a marker band for needle positioning during the procedure.

In one embodiment, the needle devices can deliver very small and accurate amount of the formulation, such as in the range of 0.01 mL to 10.0 mL, preferably 0.05 mL to 5.0 mL, most preferably 0.1 mL to 1.0 mL. Because of the micro-dosage and accurate control of the treatment volume/dose, multiple treatments in the same location/area may be used to provide a more optimal treatment outcome. As shown in FIG. 3, the triple-needle balloon catheter 108 is placed in the main renal artery 106 with balloon and needles 110 deployed; the perivascular infusion volume of a formulation can be 0.6 mL, for example. After needle retraction and balloon deflation, the catheter can continue to advance to extra-renal artery 502 (branch renal artery) and to deploy the needles and to infuse another treatment dose like 0.3 mL to its outer wall area (adventitia). If both extra-renal arteries are accessible, then both arteries can be treated with equal amount dose or different dosages. For achieving similar dosage per area in the treatment, in considerations of the surface area differences of the arterial outer wall and closer distance to kidney organ for extra-renal artery than for main renal artery, the dosage of the extra-renal artery treatment may be less than that of the main renal artery treatment. The dosage relationship between the dose to the main renal artery (DOSEₘ) and the extra-renal artery (branch) (DOSE_{b}) can be:

DOSE_{b}=DOSEₘ*(D_{b}/Dₘ)

where Dₘ is the diameter of main artery, and D_{b} is the diameter of the branch of the artery. For example, when the main artery diameter is 6 mm and the branch artery is 3 mm,

DOSE_{b}=DOSEₘ*(D_{b}/Dₘ)=DOSEₘ*(½) or =0.5 DOSEₘ

If all three areas (one main and two branches) are treated, then the total dosage of the artery treatment can be 1.2 mL for one kidney.

Sometimes a main renal artery length is long enough for a device to treat twice, e.g., once at the middle and the other nearby the bifurcated area (close to the branches) of the artery. When the main renal artery is treated twice, and two branches of the artery are treated once, the total dose for one kidney side artery treatment can be 1.8 mL.

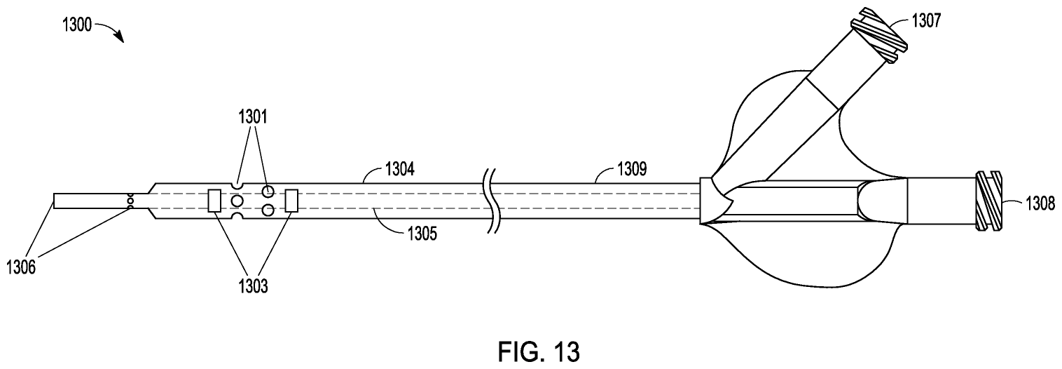

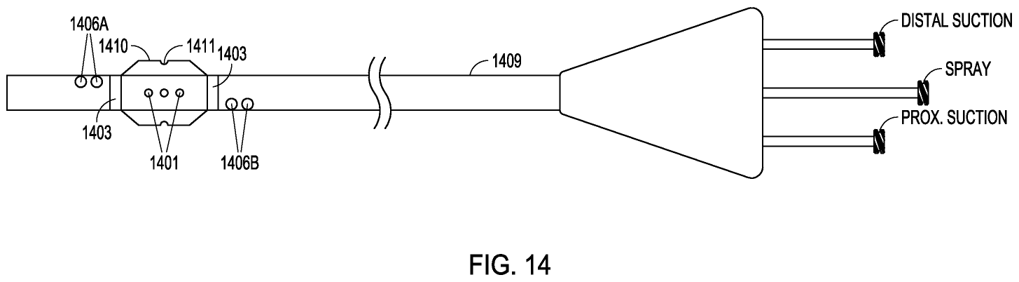

In one embodiment, shown in FIGS. 13 and 14, a spray catheter with formulation exit or spray hole(s) at its distal can be used for the delivery catheter to achieve a wider treatment area. The catheter in FIG. 13, 1300, has a co-axial design shaft 1309, in which the suction through the inner tube 1305, and the spray through the space between the inner 1305 and outer 1304 tubes. The spray hole(s) 1301 is located at the side wall of the shaft 1309, which can deliver the formulation to the vessel wall directly with good control of the spray direction and treatment area. The formulation is delivered to spray hole(s) 1301 through spray lumen 1307. If there is excess formulation in the vessel after the spray treatment, it can be removed/collected with the suction function on the catheter. The removal can be accomplished by applying a vacuum to suction port 1308 which can cause the formulation to be withdrawn through suction sites 1306. In this example, the suction opening(s) 1306 is located distal to the spray opening(s) 1301. The openings for the suction and spray can be circular or any other geometric shapes. There can be single hole or multiple holes for the suction function and the spray function and can be any combination of the number of holes between the two functions. The number of holes and the sizes of holes are independent between the suction and the spray. Ideally the hole size of the spray is smaller than that of the suction since fine mist is desired for spray. Optionally, the suction line can be connected to a vacuum pump. The spray can include two components: the liquid formulation and pressurized gas such as air or oxygen. With designated volume of the liquid agent, it is delivered via pressure gas at pre-determined pressure. Under that delivery gas pressure, the delivered formulation is in the form of fine mist that is applied on to targeted treatment area. The excess amount of the formulation after each spray can be collected/removed by the suction function on the catheter at the treatment section so the formulation can be contained in the targeted area.

The exact spray location during the procedure can be visualized via marker band under X-ray. The marker band 1303 can be located at the distal end of the catheter 1309 proximal of spray holes 1301 or dual marker bands 1303 can be located both distal and proximal of the spray holes 1301, for example. Optionally, the distal marker band is sized to occupy the space between the inner and the outer of the distal shaft, which can block the fluid path and stop the formulation at the marker band position; so, there can be less priming volume in the catheter, which can enable spray volume to be more accurate.

In another embodiment, a dual-site suction catheter can be used to help ensure that the formulation cannot run away in either direction (distally or proximally) of the spray hole(s). The example is shown in FIG. 14, in which the suction holes 1406A and 1406B are arranged distal and proximal to the spray site on the catheter. Both the distal suction hole(s) and proximal suction hole(s) can be connected through the same channel/lumen; or can be independent from each other, e.g., distal suction hole(s) and proximal suction hole(s) can be operated independently. Suction can occur at the distal position, at the proximal position, or at the both positions simultaneously. The catheter can be operated as described above.

To accommodate features in FIG. 14, a multi-lumen shaft 1409 can be used and can be, for example, a 3-lumen shaft. Each of the suction locations 1406A and 1406B has its own lumen, so the distal suction and the proximal suction can be performed independently. In one embodiment, a sleeve 1410 is placed over spray exit holes 1401 to achieve even circumferential spraying (e.g., 360°). Dual-marker bands 1403 located at the distal and proximal ends of sleeve 1410 aid in accurate positioning at the treatment site or the lesion. While FIG. 14 shows a side wall spray exit configuration, the spray exit can alternatively or additionally be placed at distal tip. With the design of the hole size 1401, the spray pattern can be a range of delivery effects (physical forms) from fine mist to coarse droplets to a jet.

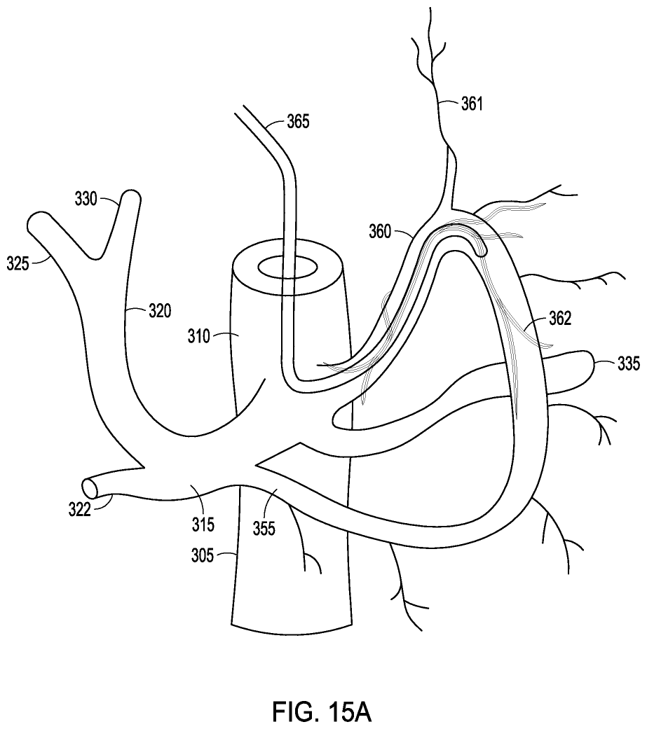

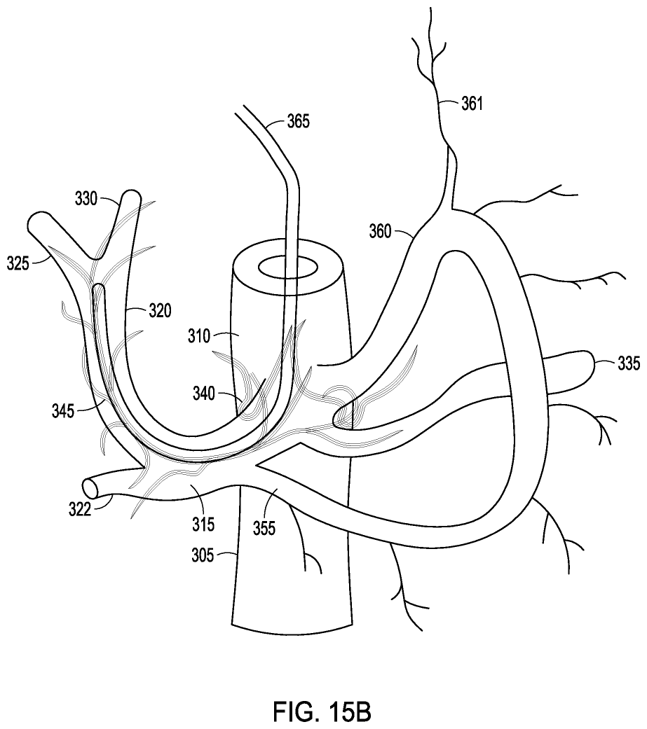

FIGS. 15A and 15B illustrate various arteries surrounding the liver and stomach as well as the various nerve systems that innervate the liver and stomach and their surrounding organs and tissues. The arteries surrounding the liver and stomach include the abdominal aorta 305, the celiac artery 310, the common 315 and proper hepatic arteries 320, the gastroduodenal artery 322, the right 325 and left hepatic arteries 330, the splenic artery 335, and esophageal branches 361. The various nerves that innervate the liver and stomach and their surrounding organs and tissues include the celiac 340 and hepatic plexuses 345 (only shown in FIG. 15B). Blood supply to the liver is pumped from the heart into the aorta and then down through the abdominal aorta 305 and into the celiac artery 310. From the celiac artery 310, blood travels through the common hepatic artery 315, into the proper hepatic artery 320, then into the liver through the right 325 and left hepatic arteries 330. The common hepatic artery 315 branches off from the celiac trunk and gives rise to gastroduodenal arteries. The nerves innervating the liver include the celiac plexus 340 and the hepatic plexus 345. The celiac plexus 340 wraps around the celiac artery 310 and continues into the hepatic plexus 345, which wraps around the proper 320 and common hepatic arteries 315, and/or continue on to the right 325 and left hepatic arteries 330. In some anatomies, the celiac 340 and hepatic plexuses 345 adhere tightly to the walls of the arteries, supplying the liver with blood, thereby rendering intra-to-extra-vascular neuromodulation particularly advantageous. In several embodiments, the average thickness of vessels (e.g., the hepatic artery) ranges from about 0.1 cm to about 0.25 cm. In some embodiments, the formulation and/or energy may be delivered to the inner wall of the target vessel or target nerves. Intravascular delivery may be employed, because the nerves tightly adhere to the outer walls of the arteries, thus supplying blood to the liver (e.g. in the case of the hepatic artery branches). In some embodiments, extraluminal/extravascular delivery is desired and can be accomplished with any of the various needle catheters disclosed herein.

The arteries surrounding the stomach include the abdominal aorta 305, the celiac artery 310, the right 355 and left gastric arteries 360, and the esophageal branches 361. Blood supply to the stomach is pumped from the heart into the aorta and then down through the abdominal aorta 305 and into the celiac artery 310. From the celiac artery 310, blood travels through the right gastric artery 355 and left gastric artery 360, the esophageal branches 361, and into the stomach. Left gastric artery 360 includes left gastric artery plexus 362 (only shown in FIG. 15A).

With continued reference to FIG. 15B, the hepatic plexus 345 is the largest offset from the celiac plexus 340. The hepatic plexus 345 is believed to primarily carry afferent and efferent sympathetic nerve fibers, the stimulation of which can increase blood glucose levels by a number of mechanisms. For example, stimulation of sympathetic nerve fibers in the hepatic plexus 345 can increase blood glucose levels by enhancing hepatic glucose production, or by reducing hepatic glucose uptake. Disruption of sympathetic nerve signaling in the hepatic plexus 345 can, therefore, alter levels of blood glucose.

The embodiment shown in FIG. 15B can be an embodiment of a balloon needle delivery catheter positioned within the hepatic artery for treatment of diabetes. The embodiment shown in FIG. 15A can be an embodiment of a triple-needle balloon delivery catheter positioned within the left gastric artery for treatment of obesity and/or diabetes. The access to these arteries is very similar to the access to renal arteries so the procedure described in previous sections is applicable in these embodiments.

A safe and effective amount of formulation can be applied in order to satisfactorily and beneficially injure tissues such as nerves. In general, the dose amount correlates with the degree of injury to the tissue. In some embodiments, an effective formulation dose ranges from 0.2 microliters to 200 milliliters. These dosing limitations are not defined however, as other delivery parameters (e.g., delivery rate, duration, and the like) may call for different doses to accomplish the ultimate injury benefit.

Treatment times can vary depending on the volume of the tissue mass to be treated, and the intended degree of injury to the target tissue. Treatment times can vary from about 2 seconds to about 60 minutes. In some embodiments, for inducing injury to relieve symptoms, the safe and effective treatment time ranges from about 4 seconds to about 30 minutes.

The delivery rate can be set by regulating the delivery system. Once the user establishes the delivery rate, formulation resources can determine the amount of pressure necessary to deliver the vapor or liquid at the desired rate.

In one embodiment, the method for treatment of hypertension includes inserting a delivery catheter percutaneously into the renal artery adjacent to the nerves; using the catheter to infuse the formulation described above and/or energy to the tissue of the body lumen adjacent to the nerves, wherein the amount of the formulation and/or energy delivered is effective to beneficially injure or damage the nerves; and withdrawing the delivery catheter from the body lumen. The injury or damage to the tissues can relieve disease symptoms, such as by lowering blood pressure. The purpose of the heat/energy can be to enhance the injury/damage effect by accelerating the reaction rate between the formulation and nerves. Potential formulations include gases, vapors, liquids, solutions, emulsions, suspensions of one or more ingredients, and combinations thereof. If the formulation includes vapors of one or more ingredients, the heat can be generated by condensation of the vapors into liquids in the tissue. If the formulation includes liquids or solutions, the heat can be transferred from the high-temperature formulations that exceed body temperature. The formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue adjacent to the nerves may be lower than the formulation temperature and higher than body temperature. The temperature of the treated tissue adjacent to the nerves can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more. The formulation infusion pressure can be 0.1 to 14 atm, preferably from 3 to 10 atm, most preferably from 4 to 8 atm.

In one embodiment, the method for treatment of asthma includes inserting a delivery catheter into the airways adjacent to the nerves; using the catheter to infuse the formulation described above and/or energy to the tissue of the airway adjacent to the nerves, wherein the amount of the formulation and/or energy delivered is effective to beneficially injure or damage the nerves; and withdrawing the delivery catheter from the body lumen. The injury or damage to the nerves can relieve disease symptoms, such as by reliving shortness of breath. The purpose of the heat/energy can be to enhance the injury/damage effect by accelerating the reaction rate between the formulation and nerves. Potential formulations include gases, vapors, liquids, solutions, emulsions, suspensions of one or more ingredients, and combinations thereof. If the formulation includes vapors of one or more ingredients, the heat can be generated by condensation of the vapors into liquids in the tissue. If the formulation includes liquids or solutions, the heat can be transferred from the high-temperature formulations that exceed body temperature. The liquid formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue adjacent to the nerves may be lower than the formulation temperature and higher than body temperature. The temperature of the treated tissue adjacent to the nerves can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more. The formulation infusion pressure should range from 0.1 to 14 atm, preferably from 3 to 10 atm, most preferably from 4 to 8 atm.

Alternatively, asthma can be treated with the spray catheters without balloons. In one embodiment, the method for treatment of asthma includes inserting a delivery catheter into the airways adjacent to the nerves; using the catheter to infuse the formulation described above and/or energy to the tissue of the airway adjacent to the nerves, wherein the amount of the formulation and/or energy delivered is effective to beneficially injure or damage the nerves; and withdrawing the delivery catheter from the body lumen. The injury or damage to the nerves can relieve disease symptoms, such as by relieving shortness of breath. The benefits of this spray method are that it is simple, fast and able to treat small diameter airways. Potential formulations include gases, vapors, liquids, solutions, emulsions, suspensions of one or more ingredients, and combinations thereof. With liquid and gas formulation, a fine mist can be preferred for delivery. The diameter of formulation exiting the spray hole can be 0.5 mm or less, more preferably 0.4 mm or less. Delivery gas pressure can be preferred to be less than 8 atm, more preferably at 4 atm or less.

In one embodiment, the method for treatment of a COPD and/or asthma includes inserting a delivery catheter into the airway adjacent to the nerves; using the catheter to infuse the formulation described above and/or heat to the tissue of the body lumen adjacent to the nerves, wherein the amount of the formulation and/or heat delivered is effective to beneficially injure or damage the nerves; and withdrawing the delivery catheter from the airway. The injury or damage to the nerves can relieve disease symptoms, such as by relieving COPD/asthma symptoms. The purpose of the heat/energy can be to enhance the injury/damage effect by accelerating the reaction rate between the formulation and nerves. Potential formulations include gases, vapors, liquids, solutions, emulsions, and suspensions of one or more formulations. If the formulation includes vapors of one or more ingredients, the heat can be generated by condensation of the vapors into liquids. If the formulation includes liquids or solutions, the heat can be transferred from the high-temperature formulations that exceed body temperature. The formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue adjacent to the nerves may be lower than the formulation temperature and higher than body temperature. The temperature of the treated tissue adjacent to the nerves can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The formulation infusion pressure and/or the balloon inflation pressure can be 0.1 to 14 atm, preferably from 3 to 10 atm, most preferably from 4 to 8 atm.

Alternatively, COPD can be treated with the spray catheters as well, in which the same method described herein for asthma treatment can be used. The method also includes the combination use of balloon catheter and spray catheter in the same procedure, which may result in maximized denervation effect.

In one embodiment, the method for treatment of severe emphysema with a lung volume reduction includes inserting a delivery catheter into the airway adjacent to the targeted area; using the catheter to spray the formulation described above and/or energy to completely destroy the targeted lung tissue, wherein the amount of the formulation and/or energy delivered is effective to beneficially damage the tissues; and withdrawing the delivery catheter from the airway. The damage to the tissues can relieve disease symptoms, such as emphysema symptoms. The formulation and/or energy can be delivered at distal tip front, or side wall position, or the combination of both.

In one embodiment, the method for treatment of diabetes and/or nonalcoholic fatty liver disease (NAFLD) and/or nonalcoholic steatohepatitis (NASH) includes inserting a delivery catheter percutaneously into the hepatic arteries adjacent to the nerves, specifically the hepatic celiac artery, hepatic common artery, proper hepatic arteries, or the left and right hepatic arteries; using any of the catheters described herein to infuse the formulation described above and/or energy to the tissue of the body lumen adjacent to the nerves or directly to the nerves, wherein the amount of the formulation and/or energy delivered is effective to beneficially injure or damage the nerves; and withdrawing the delivery catheter from the body lumen. The purpose of the energy/heat can be to enhance the injury/damage effect by accelerating the reaction rate between the formulation and nerves. Potential formulations include gases, vapors, liquids, solutions, emulsions, suspensions of one or more ingredients, and combinations thereof. If the formulation includes vapors of one or more ingredients, the heat can be generated by condensation of the vapors into liquids in the tissue. If the formulation includes liquids or solutions, the heat can be transferred from the high-temperature formulations that exceed body temperature. The formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue adjacent to the nerves may be lower than the formulation temperature and higher than body temperature. The temperature of the treated tissue adjacent to the nerves can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more. The formulation is delivered via needles to the perivascular area for denervation with the formulation volume ranging from 0.1 mL to 1.0 mL, preferably from 0.3 mL to 0.8 mL, most preferably from 0.4 mL to 0.7 mL.

In one embodiment, a method for reduction of fast blood glucose, HbA1c, glucagon, epinephrine, norepinephrine, cortisol, or growth hormone of diabetes patients can include inserting a delivery catheter percutaneously into the hepatic arteries adjacent to the nerves, specifically the hepatic celiac artery, hepatic common artery, proper hepatic arteries, and/or the left and right hepatic arteries; using any of the catheters described herein to infuse the formulation described above and/or energy to the tissue of the body lumen adjacent to the nerves or directly to the nerves, wherein the amount of the formulation and/or energy delivered is effective to beneficially injure or damage the nerves; and withdrawing the delivery catheter from the body lumen. The purpose of the heat/energy can be to enhance the injury/damage effect by accelerating the reaction rate between the formulation and nerves. Potential formulations include gases, vapors, liquids, solutions, emulsions, suspensions of one or more ingredients, and combinations thereof. If the formulation includes vapors of one or more ingredients, the heat can be generated by condensation of the vapors into liquids in the tissue. If the formulation includes liquids or solutions, the heat can be transferred from the high-temperature formulations that exceed body temperature. The formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue adjacent to the nerves may be lower than the formulation temperature and higher than body temperature. The temperature of the treated tissue adjacent to the nerves can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The formulation is delivered via needles to the perivascular area for denervation with the formulation volume ranging from 0.1 mL to 1.0 mL, preferably from 0.3 mL to 0.8 mL, most preferably from 0.4 mL to 0.7 mL.

In one embodiment, the method for treatment of obesity and/or diabetes includes inserting any of the delivery catheters described herein into the left and/or right gastric arteries adjacent to the stomach and esophageal nerves; using the catheter to infuse the formulation described above and/or energy to the tissue of the gastric arteries or to outside the arteries adjacent to the nerves, wherein the amount of the formulation and/or energy delivered is effective to beneficially injure or damage the nerves; and withdrawing the delivery catheter from the gastric arteries. Potential formulations include gases, vapors, liquids, solutions, emulsions, suspensions of one or more ingredients, and combinations thereof. If the formulation includes vapors of one or more ingredients, the heat can be generated by condensation of the vapors into liquids. If the formulation includes liquids or solutions, the heat can be transferred from the high-temperature formulations that exceed body temperature. The liquid formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue adjacent to the nerves may be lower than the formulation temperature and higher than body temperature. The temperature of the treated tissue adjacent to the nerves can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more. The formulation infusion pressure and/or the balloon inflation pressure should range from 0.1 to 14 atm, preferably from 3 to 10 atm, most preferably from 4 to 8 atm.

In one embodiment, the method for treatment of obesity includes inserting any of the delivery catheters described herein into or outside of the digestive lumen adjacent to the nerves; using the catheter to infuse the formulation described above and/or energy to the tissue of the digestive lumen, wherein the amount of the formulation and/or energy delivered is effective to beneficially injure or damage the tissue; and withdrawing the delivery catheter from the digestive lumen. Potential digestive lumens for this embodiment include the esophagus, the stomach, the duodenum, the jejunum, the small and large intestines, and the colon. The purpose of the heat/energy can be to enhance the injury/damage effect by accelerating the reaction rate between the formulations and nerves. Potential formulations include gases, vapors, liquids, solutions, emulsions, suspensions of one or more ingredients, and combinations thereof. If the formulation includes vapors of one or more ingredients, the heat can be generated by condensation of the vapors into liquids. If the formulation includes liquids or solutions, the heat can be transferred from the high-temperature formulations that exceed body temperature. The liquid formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue adjacent to the nerves may be lower than the formulation temperature and higher than body temperature. The temperature of the treated tissue adjacent to the nerves can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more.

In one embodiment, the method for treatment of obesity and diabetes includes inserting any of the delivery catheters described herein into the left and/or right gastric arteries adjacent to the stomach and esophageal nerves; using the catheter to infuse the formulation described above and/or energy to the tissue of or outside the gastric arteries adjacent to the nerves, wherein the amount of the formulation and/or energy delivered is effective to beneficially injure or damage the nerves; and withdrawing the delivery catheter from the gastric arteries. Potential formulations include gases, vapors, liquids, solutions, emulsions, suspensions of one or more ingredients, and combinations thereof. If the formulation includes vapors of one or more ingredients, the heat can be generated by condensation of the vapors into liquids. If the formulation includes liquids or solutions, the heat can be transferred from the high-temperature formulations that exceed body temperature. The liquid formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue adjacent to the nerves may be lower than the formulation temperature and higher than body temperature. The temperature of the treated tissue adjacent to the nerves can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more.

In one embodiment, the method for treatment of urological diseases and/or benign prostate hyperplasia (BPH) includes inserting a delivery catheter into the urological lumen; using any of the catheters described herein to infuse the formulation described above and/or energy to the or outside of the lumen of a urological tissue, e.g. the prostate, urethra, and ureter, wherein the amount of the formulation and/or energy delivered is effective to beneficially injure or damage the tissue; and withdrawing the delivery catheter from the urological lumen. The purpose of the heat/energy can be to enhance the injury/damage effect by accelerating the reaction rate between the formulation and nerves. The formulations include one of gases, vapors, liquids, solutions, emulsions, suspensions of one or more ingredients, and combinations thereof. If the formulation includes vapors of one or more ingredients, the heat can be generated by condensation of the vapors into liquids. If the formulation includes liquids or solutions, the heat can be transferred from high-temperature formulations that exceed body temperature. The liquid formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue adjacent to the nerves may be lower than the formulation temperature and higher than body temperature. The temperature of the treated tissue adjacent to the nerves can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more. The formulation infusion pressure and/or the balloon inflation pressure can be 0.1 to 14 atm, preferably from 3 to 10 atm, most preferably from 4 to 8 atm.

In one embodiment, the method for treatment of cancers or tumors includes inserting a needle or needle-based catheter percutaneously, trans-orally, or trans-luminally into the cancers or tumors under imaged guidance; using the catheter to infuse the formulation described above and/or energy to the cancer tissues of the human body, wherein the amount of the formulation and/or energy delivered is effective to beneficially injure, damage or eliminate the cancer tissues; and withdrawing the delivery catheter from the body. The injury, damage, or elimination of the cancer tissues can relieve disease symptoms, such as by shrinking or eliminating tumors. Potential imaging guides include ultrasound, X-ray, CT scan, NMR imaging, and scopes. Relevant cancers include adrenal, bladder, cervical, colon, esophageal, gallbladder, kidney, liver, lung, ovarian, pancreatic, prostatic, rectal, stomach, and uterine. The purpose of the heat/energy can be to enhance the injury/damage/elimination effect by accelerating the reaction rate between the formulation and cancer tissues. The formulations include one of gases, vapors, liquids, solutions, emulsions, suspensions of one or more ingredients, and combinations thereof. If the formulation includes vapors of one or more ingredients, the heat can be generated by condensation of the vapors into liquids in the tissue. If the formulation includes liquids or solutions, the heat can be transferred from the high-temperature formulations that exceed body temperature. The formulation temperature can be −40 to 140° C., −30 to 100° C., −30 to 80° C., or −40° C. or less, or less than, equal to, or greater than −30° C., −20, −10, −5, 0, 5, 10, 20, 30, 40, 50, 60, 70, 80, 90, 100, 110, 120, 130, or 140° C. or more. The temperature of the treated tissue may be lower than the formulation temperature and higher than body temperature. The temperature of the treated tissue can be −40 to 100° C., −30 to 90° C., −20 to 80° C., or −40° C. or less, or less than, equal to, or greater than −30° C., −20, −10, −5, 0, 5, 10, 20, 30, 40, 50, 60, 70, 80, 90, or 100° C. or more.

A method for treatment of myocardial infarction, atherosclerosis, coronary artery disease (CAD), peripheral vascular disease (PAD), rheumatoid arthritis, or cancer due to the immune response to inflammatory conditions can include inserting a delivery catheter percutaneously into the splenic arteries adjacent to the nerves; using any one of the catheters described herein to infuse the formulation described above and/or energy to the tissue of the body lumen adjacent to the nerves or directly to the nerves, wherein the amount of the formulation and/or energy delivered is effective to beneficially injure or damage the nerves; and withdrawing the delivery catheter from the body lumen. The formulation can be delivered via needles to the perivascular area for denervation with the formulation volume ranging from 0.1 mL to 1.0 mL per application, or 0.3 mL to 0.8 mL, or 0.4 mL to 0.7 mL.

A method for treatment of myocardial infarction, atherosclerosis, coronary artery disease (CAD), peripheral vascular disease (PAD), due to hypercholesterolemia conditions can include inserting a delivery catheter percutaneously into the splenic arteries adjacent to the nerves; using any one of the catheters described herein to infuse the formulation described above and/or apply energy to the tissue of the body lumen adjacent to the nerves or directly to the nerves, wherein the amount of the formulation and/or energy delivered is effective to beneficially injure or damage the nerves; and withdrawing the delivery catheter from the body lumen. The formulation can be delivered via needles to the perivascular area for denervation with the formulation volume ranging from 0.1 mL to 1.0 mL per application, or 0.3 mL to 0.8 mL, or 0.4 mL to 0.7 mL.

Hypertension and hypercholesterolemia can be predispositions to vascular diseases such as coronary heart disease, but the two acting in concert alter risk substantially because their combined effects are considered to be multiplicative rather than additive. Persons with a combination of risk factors are, therefore, at particularly high risk of coronary heart disease. The impaired endothelium-dependent vascular relaxation in patients with essential hypertension may be associated with hypercholesterolemia; also heightened sympathetic activity may be related to hyperlipidemia in hypertension. For example, the positive association of serum triglyceride levels with blood pressure is significant especially for the persons with high body mass index (BMI>24). Embodiments of the present invention can not only to reduce the blood pressure but also to reduce the cholesterols levels. The biological interrelations between blood pressure and atherogenic blood lipid fractions and the pathophysical factors underlying these interrelations may influence the mechanisms whereby hypertension is associated with increased risk of coronary heart disease. Therefore, in the outcomes of various embodiments of the method of treatment, either reduction in blood pressure or in cholesterols levels, or reduction in both blood pressure and cholesterols levels, can be beneficial to the risk reduction of coronary artery disease (CAD) and peripheral vascular disease (PAD).

A method for treating a disease with a needle-based balloon infusion system, wherein the needle-based balloon infusion system includes a needle-based balloon delivery catheter, a guiding wire, an ablation agent, saline or heparinized saline or therapeutic medicine, and a set of fluid storages, can include: 1) inserting a needle-based balloon delivery catheter into a body lumen, wherein the needle-based balloon delivery catheter includes a distal head, at least one marker band, at least one needle, at least one needle exit hole, at least one flushing hole, a flushing lumen, a flushing port, a guide wire lumen, at least one centering balloon at approximately distal end of the catheter, one inflating lumen, one inflating port, one ablating port, a needle movement shaft, and a needle movement controller; wherein the needle lumen is prefilled with ablation agent, the flushing lumen is prefilled with flushing medium prior to the inserting; 2) inflating the centering balloon to center the delivery catheter shaft in body lumen; 3) deploying the at least one needle into a wall, outside the wall, or inside the wall of the body lumen; 4) infusing a formulation through the at least one needle, wherein the amount of the formulation delivered is effective to injure or damage target tissue to relieve a disease symptom; 5) optionally removing the formulation from the tissue; 6) retracting the needles into the delivery catheter and deflating the centering balloon; 7) flushing the flushing lumen prior to the balloon deflation or prior to inserting to next body lumen to prevent needle lumen blocking and blood clot forming; 8) inserting a needle-based balloon delivery catheter into next body lumen; 9) repeating 2) to 8) until all the targeted body lumen are treated; and 10) withdrawing the delivery catheter from the body lumen. The disease can be hypertension, pulmonary hypertension, diabetes, obesity, metabolic syndrome, heart failure, myocardial infarction, atherosclerosis, coronary artery disease (CAD), peripheral vascular disease (PAD), end-stage renal disease, a digestive disease, nonalcoholic fatty liver disease (NAFLD), nonalcoholic steatohepatitis (NASH), urological disease, cancers, tumors, pain, rheumatoid arthritis (RA), asthma, chronic obstructive pulmonary disease (COPD), or a combination thereof. The ablation agent can be one of ethanol, dehydrated ethanol, acetic acid, diluted acetic acid. The flushing medium can include saline or heparinized saline or a neutralized agent to the ablation agent. The body lumen can include renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, urological lumens, or a combination thereof.

A method of using a needle-based balloon delivery catheter can include: prefilling the needle lumen with an ablation agent, prefilling the flushing lumen with a flushing medium, and inserting the catheter into the guiding catheter over the guidewire. Once reaching the treatment site, the balloon can be inflated to the size of the vessel to center the needle head, then the needles can be advanced to the treatment position, and the ablation agent can be infused; after the infusion, the needles can be withdrawn and retracted back into the distal head, flushing fluid can be injected to keep the needle compartment/hole of the distal head and the needle tip/opening clean and away from the direct contact of blood. The balloon can be deflated and the catheter can be withdrawn from the treatment site and/or moved to another treatment site.

In some embodiments, when a targeted vessel is long and needs to be treated more than once, a distance between the treatment sites can be used to separate the infused ablation agent. The separated distance between treatment sites can be from 5 mm to 30 mm, such as 5 mm to 15 mm.

The dosage of the ablation agent can be 0.1 mL, 0.2, 0.3, 0.4, 0.5, 0.6, 0.8, 1.0, or 1.2 mL for each application/treatment for a main vessel such as artery. If the treatment is performed more than once, the total dosage (volume) for the particular main vessel (length) can be 0.3 mL, 0.4, 0.5, 0.6, 0.7, 0.8, 0.9, 1.0, 1.1, 1.2, 1.3, 1.4, 1.5, 1.6, 1.7, 1.8, 1.9, 2.0, or 2.4, or up to 3.6 mL. For a branch vessel or artery, the dosage of each application can be 0.1 mL, 0.2, 0.3, 0.4, 0.5, or 0.6 mL. If the treatment is performed more than once, the total dosage for this branch can be 0.2 mL, 0.4, 0.6, 0.8, 1.0, or 1.2 mL.

Various embodiments of the present invention provide a needle-based balloon delivery catheter for delivery of materials to a target tissue in the body lumen of a patient. The delivery catheter can include a catheter shaft with a proximal and distal end. The delivery catheter can include at least one marker band located near the distal end of the shaft. The delivery catheter can include at least one needle (e.g., 3 needles), wherein each needle is situated in a needle lumen. The needle lumen can be open to the outside of the catheter shaft via at least one needle exit hole. The delivery catheter can include a flushing port at the proximal end of the shaft in fluid communication with a flushing lumen. The flushing lumen is in fluid communication with a distal end of the needle lumen. The flushing port can be in fluid communication with the needle exit hole through the flushing lumen. The delivery catheter can include a guide wire lumen which extends through at least the distal end of the shaft. The delivery catheter can include at least one balloon adjacent to the distal end of the catheter. The delivery catheter can include an inflation lumen. The delivery catheter can include an inflation port in fluid communication with the inflation lumen and in fluid communication with an interior of the balloon. The balloon is inflatable via the inflation port through the inflation lumen and can approximately center the distal end of the catheter shaft in a body lumen during inflation. The delivery catheter can include an ablation or denervation port at the proximal end of the shaft. The ablation or denervation port is in fluid communication with the at least one needle for supplying ablation energy or formulation to the at least one needle. The delivery catheter can also include a needle movement controller in electrical or mechanical communication with the at least one needle. The needle movement controller can deploy the at least one needle into the body lumen, into a wall of the body lumen, or outside of the body lumen. Deployment of the needle through the needle-exit opening can allow the formulation to penetrate into the wall of the body lumen at a pressure higher than that of the body lumen. The target tissue can be target tissue of renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, urological lumens, or a combination thereof. The formulation pressure during infusion can be any suitable pressure, and the balloon inflation pressure can be any suitable pressure. For example, the pressure of the formulation infusion can range from 0.1 to 14 atm. The balloon inflation pressure can range from 0.1 to 14 atm.

### EXAMPLES

Various embodiments of the present invention can be better understood by reference to the following Examples which are offered by way of illustration. The present invention is not limited to the Examples given herein.

### Example 1. Pre-Clinical Trial

A porcine animal weighing of 47 kg was anesthetized with isoflurane, and one side of its renal artery was ablated with ethanol using a triple-needle single-balloon catheter while the contralateral renal artery served as a control. The catheter needles span diameter was about 10 mm. The catheter balloon is a low pressure and compliant balloon with a diameter of 7 mm at 1 atm, which functions to center the distal shaft without over-stretching the artery. Optionally, the balloon can be inflated with a syringe without using an inflation device. Because of the balloon characteristics, the balloon can be used for any diameter artery (under or above the balloon diameter). Before insertion into the artery, the catheter was pre-filled with the liquid formulation until overflow occurred from the needles (with needles out), then the needles were retracted and the syringe with predetermined treatment volume was connected to the catheter. Using a standard renal access procedure, the triple-needle balloon delivery catheter was placed into the targeted renal arteries of the main and extra-renal arteries (branches) in sequence over a guide wire. Upon reaching the targeted ablation site, the balloon was inflated, and then the needles were deployed, and absolute ethanol at room temperature was infused to the adventitial and peri-adventitial space of renal artery. The infusion volume was 0.6 mL for the main artery and 0.3 mL for the branch per application with the 1 mL syringe. The infusion time was within 5 seconds for both application volume. During the balloon inflation, the balloon diameter was monitored by fluoroscopy to have about 1:1.1 ratio (slightly bigger) compared to the diameter of surrounding artery. By the end of the treatment time, the needles were retracted into the catheter first, then the balloon was deflated, and the catheter was ready to be withdrawn or to be placed into another artery site if required for the next treatment. In this example, a total of 4 treatments were done: 2 in main artery and 1 for each branch, and a total volume of formulation of 1.8 mL was used.

Post-ablation renal angiograms were obtained to exam if occurrences of any vessel spams, stenosis, and other abnormalities. There were no significant renal arterial spasms during and after the infusion treatment.

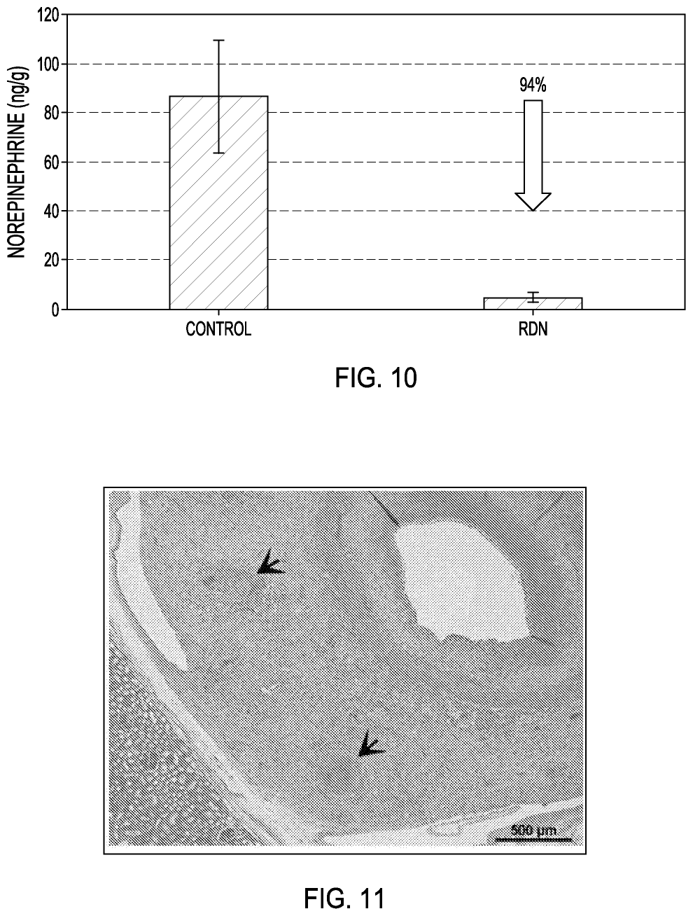

The animal was euthanized two weeks after treatment, the gross necropsy showed all treatment related organs were normal, and renal tissue samples were obtained from cranial (n=3), middle (n=3), and caudal (n=3) of the renal cortex to determine the renal tissue norepinephrine (NE) content using known HPLC methods. Norepinephrine is a neurotransmitter used by the sympathetic nervous system whose levels serve as a standard measurement for renal denervation. Comparing to the NE concentration of untreated side (the control), the NE concentration of the treated side is lower or much lower after denervation, as shown in FIG. 10. Ethanol ablation of the renal artery resulted in a 94% reduction in renal norepinephrine concentration (the average NE content: control: 87 ng/g vs. treated: 4.9 ng/g). The percentage range of individual NE reduction, calculated from the similar tissue location between the control and the treated, is from 89% to 97.5%. Renal arteries and surrounding tissues were collected for histopathologic evaluation as well.

Not only was the NE content reduced after ethanol ablation, but histopathologic evaluation also demonstrated renal nerve injury, as shown in FIG. 11 by nerves (see the arrows) surrounded by mild fibrosis within the outer margins of the tunica adventitia.

### Example 2. Pre-Clinical Trial

Two 46 kg porcine animals were treated with hepatic artery ethanol ablation, using a formulation that was absolute ethanol at room temperature and using the same kind of triple-needle single-balloon catheter as used in Example 1. Before insertion into the artery, the catheter was pre-filled with the liquid formulation until overflow occurred from the needles (with needles out), then the needles were retracted and the syringe with predetermined treatment volume was connected to the catheter. A standard hepatic artery access procedure was performed. Each hepatic artery was treated two times that were evenly spaced out along the artery length and the infusion volume was 0.6 mL per application with 1 mL syringe and the infusion time was less than 5 seconds. The inflated balloon diameter was monitored to be less than 1:1.1 expanding ratio to the artery, and the inflation was performed with a syringe. Hepatic angiograms showed no significant arterial spasms during and after the infusion treatment in both porcine animals of this study. As a control, an untreated similar body weight porcine animal was used for NE reduction calculation and histopathologic evaluation comparison.

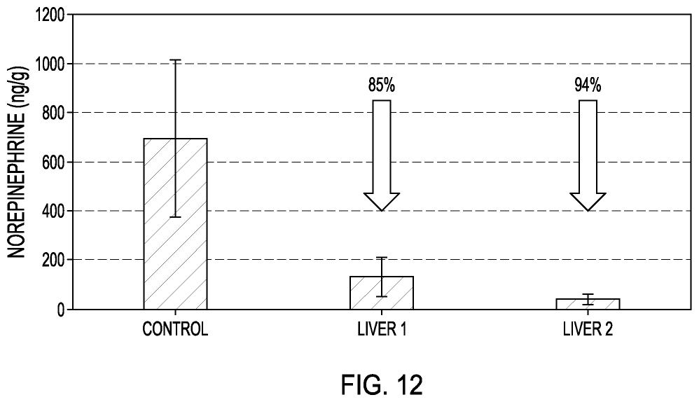

The two animals were euthanized two weeks after the treatment; again, the gross necropsy showed all treatment related organs were normal, and liver tissue samples were obtained from right lateral lobe (n=2), right medial lobe (n=2), left medial lobe (n=2), left lateral lobe (n=2) and caudate lobe (n=2) to determine the liver tissue norepinephrine (NE) content using known HPLC methods. The NE reduction was calculated based upon the data collected from the tissues of similar location between the treated and the control. The average liver NE concentration reductions of the two animals were 85% and 94%, as shown in FIG. 12. The individual calculated percentage range was from 55% to 95% for one animal, and from 86% to 97.5% for the other animal. As in the renal ablation study, the arteries and surrounding tissues were collected for histopathologic evaluation.

The above pre-clinical findings demonstrate that ethanol treatment is effective and safe.

### Example 3. Clinical Trial, Metabolic Syndrome

In one example, human male patient A was 53 years old with metabolic syndrome, who had had hypertension for 10 years and taken 2 antihypertensive drugs, had type 2 diabetes for 2 years and taken 2 T2DM (Type 2 diabetes) medications, and had obesity for 8 years. His triglycerides level was 336 mg/dL before the procedure. The targeted arteries for his treatment were renal arteries, hepatic artery, splenic artery, and the left gastric artery.

In the procedure, modest sedation per the institution's standard practice was used and no general anesthesia was used. The arteries were engaged using a 7F guiding catheter, introduced via the femoral artery. Angiography was performed of the arteries before intervention. All four arteries were treated in the same procedure. The treatment started at upper body arteries in the order of splenic, hepatic, left gastric, left renal, and right renal arteries. After the initial angiography survey of the hepatic artery, splenic artery and left gastric artery, the rapid-exchange version of the triple-needle single-balloon catheter was advanced through the guiding catheter and into the artery over a guiding wire. Before insertion into the artery, the catheter was pre-filled with the liquid ablation formulation (dehydrated ethyl alcohol) until overflow occurred from the needles (with needles out), then the needles were retracted and the syringe with predetermined treatment volume was connected to the catheter.

The maximum catheter needle span diameter was about 12 mm, and the span diameter was adjustable per artery diameter. The catheter balloon was a low pressure and compliant balloon with a diameter of 7 mm at around 1 atm. The function of this balloon was to center the distal head/shaft without over-stretching the artery. The balloon was inflated with a 30 cc syringe with a stopcock (without using an inflation device) to have gentle control of the balloon diameter.

The triple-needle balloon delivery catheter was placed into the targeted splenic artery under fluoroscopic guidance. Upon reaching the targeted ablation site, the balloon was inflated, and then the needles were deployed, and absolute ethanol at room temperature was infused to the adventitial and peri-adventitial space of splenic artery. The infusion volume was 0.6 mL per application with the 1 mL syringe. The infusion time was within 10 seconds for each application. During the balloon inflation, the balloon diameter was monitored by fluoroscopy to have about 1:1.1 ratio (slightly bigger) compared to the diameter of surrounding artery. At the end of the treatment time, the needles were retracted into the catheter, then the balloon was deflated, and the catheter was moved to the second treatment site, which was about 15 mm away from the first treatment site, in the same splenic artery. The treatment steps were repeated for the second treatment (balloon inflation, needle deployment, infusion ablation, needle withdrawal, balloon deflation). The total volume of the alcohol used in the two treatments was 1.2 mL for the splenic artery.

The same treatment method and procedure steps as in the treatment of the splenic artery were used for common hepatic artery ablation. The catheter was replaced with a fresh catheter for the common hepatic artery ablation. There were two treatments total at about 15 mm apart of each other (between the treatment sites) for the common hepatic artery with the same ablation dosage of 0.6 mL. The total volume of the alcohol used for the hepatic artery ablation was 1.2 mL. After the hepatic treatment, the catheter was placed into the left gastric artery for next ablation treatment. Again, the same treatment method and procedure was used, with the exception that one ablation treatment of 0.6 mL of the alcohol was applied for the left gastric artery. Angiography was performed after the withdrawal of the triple-needle balloon delivery catheter.

After the ablation treatments of the three arteries above, angiography was performed of the renal arteries before intervention. The suitable branches and main arteries were assessed and predetermined for treatment. After the assessment, the triple-needle balloon delivery catheter was advanced through the guiding catheter and over the guiding wire into the branch artery of left renal arteries and stopped just beyond the bifurcated area. The same catheter operation method and procedure steps were used, and the branch artery was ablated once with 0.3 mL of the alcohol. Upon the completion of the branch ablation, the catheter was pulled back into the main artery at a site that was just after the bifurcated area, and the site was treated with 0.6 mL of the alcohol. Then the catheter was pulled back about 10 mm away from the first treatment site in the main artery, to a second ablation site, where a second ablation treatment was applied with a volume of 0.6 mL of the alcohol. A total of 3 treatments were done on the left renal artery: 2× in main artery and 1× in branch artery, and the total volume of the alcohol used was 1.5 mL.

The same method and procedure steps were repeated for the right renal artery. After the initial angiography and the assessment of the right renal artery, the triple-needle balloon delivery catheter was advanced through the guiding catheter and over the guiding wire into the branch artery #1. The branch #1 was ablated twice at about 10 mm apart between the treatment sites and using 0.3 mL of alcohol for each treatment. Then the catheter was placed into branch artery #2, which was treated twice with the alcohol volume of 0.3 mL for each treatment. After the treatments of the both branches, the catheter was pulled back into the main artery until its needles were near the midpoint of the main artery, and the right main artery was ablated once with an alcohol volume of 0.6 mL. The total alcohol volume used for the right renal artery treatments was 1.8 mL as well.

Patient A had been followed up for the time points of 2 weeks (2 wks), 1 month (1 mo), and 3 months (3 mos) post-procedure and his follow-up data were listed below along with his baseline (Base) data obtained before the procedure for comparison. There were not any medication changes during follow-up period.

Body weight: base=103 kg, 2 wk=96.2 kg, 1 mo=95.6 kg, 3 mos=96.2 kg. The patient weight loss had been observed at about 7% reduction since week 3 (2 wk time point) after the procedure and the reduced body weight was maintained at 3 months follow-up.

24-Hour blood pressure, SBP/DBP: base=164/109 mmHg, 1 mo=140/87 mmHg, 3 mo=141/84 mmHg. The blood pressure data are the average value of the 24-hour ambulatory blood pressures. Both systolic (SBP) and diastolic (DBP) blood pressure drops are significant at about 14% and 20%, respectively, from the baseline; and the lowered blood pressure was maintained at 3 months follow-up.

Blood sugar level, HbA1c: base=7.5%, 3 mos=6.6%. The patient's hemoglobin A1c (HbA1c) level had meaningful improvement as well. Comparing to the baseline value, his HbA1c value change was almost −1(%) at the 3 months follow-up.

Triglycerides: base=336 mg/dL, 2 wks=160 mg/dL, 1 mo=139 mg/dL, 3 mos=167 mg/dL. Triglycerides (TG) are a type of fat (lipid) found in the blood. High triglyceride level can be a clue that the person has fatty liver disease. The improvement of patient A's triglyceride level was observed at two weeks follow-up time at about 50% reduction. The lowered and improved triglyceride level was maintained at 3 months follow-up. At 1-month follow-up, Patient A's TG level was in the normal range.

High-density lipoprotein (HDL): base=39 mg/dL, 2 wks=42 mg/dL, 1 mo=43 mg/dL, 3 mos=40 mg/dL. Patient A's high-density lipoprotein (HDL) level at baseline was just under normal range. Then at follow-ups, his HDL seemed to have some improvement and all 3 numbers were at 40 or higher. Patient A's TG/HDL ratio had significant improvement from 8.6 at baseline (>5 abnormal) to 4.2 at 3-month follow-up that was within the normal range (<5); patient A's NASH condition was meaningfully improvement if the patient had NAFLD.

The short-term outcomes of patient A were significant and very encouraging for the treatment and technology. The data indicates that the patient health improved and his diseases associated with metabolic syndrome seemed under control at 3 months follow-up.

### Example 4. Clinical Trial, Hypertension

In another example, multiple arteries/tissues were treated for hypertension. A male patient B of 50 years old had his blood pressure controlled after the ablation procedure at the 3 months follow-up time point. Patient B had 5 years of hypertension history. Patient B went through the same procedure and ablation treatment method as patient A in Example 3. The treated arteries and the ablation dosages for each treatment site are summarized in the Table 1 for patient B.

TABLE 1
Patient B treated arteries and ablation
dosages for each treatment site.

| | Artery | | | | | | |
|---|---|---|---|---|---|---|---|
| | | | | R- | R- | L- | L- |
| | | | | Renal | Renal | Renal | Renal |
| | Hepatic | Splenic | Celiac | B | M | B | M |
| # of | 2 | 2 | 1 | 1 | 2 | 1 | 2 |
| treatment | | | | | | | |
| Dosage of | 0.6 | 0.6 | 0.6 | 0.3 | 0.6 | 0.3 | 0.6 |
| each | mL | mL | mL | mL | mL | mL | mL |
| treatment | | | | | | | |

Renal B: renal branch artery; Renal M: renal main artery; R: right; and L: left.

The post-treatment data of average 24-hour ambulatory blood pressure were: base=136/79 mmHg, 1 mo=107/61 mmHg, 3-mo=115/66 mmHg. Both systolic (SBP) and diastolic (DBP) blood pressure drops are significant at about 21% and 23% respectively from the baseline at 1 month follow-up; and his improved blood pressure was maintained at 3 month follow-up time with about 15% percentage drops for both SBP and DBP from his baseline level.

### Example 5. Clinical Trial, T2DM

In another example, multiple arteries/tissues were treated for diabetes. The same patient B of above had 1 year of T2DM history and his hepatic, splenic, celiac, right renal and left renal arteries were treated for the diabetes disease. The results of the treatments in Example 4 yielded some improvements on patient B's fasting glucose value and HbA1c numbers.

The post-treatment data of fasting glucose were: base=186 mg/dL, 2 wks=127 mg/dL, 1 mo=92 mg/dL, 3 mos=110 mg/dL. The test was done in the morning before the patient had eaten. A fasting blood sugar level from 100 to 125 mg/dL (5.6 to 6.9 mmol/L) is considered prediabetes. If it is 125 mg/dl or higher, the person has diabetes. The data indicated that patient B had diabetes before the procedure. His fasting glucose level had significant improvement from his baseline after the procedure with −32% change at 2 weeks follow-up and −50% change at 1-month follow-up. At the 3 months follow-up time his lower glucose level was still maintained at the lower level of 110 mg/dL, which was −41% reduction from the baseline.

Blood sugar HbA1c data: base=8.1%, 3 mos=6.0%. The patient had his HbA1c improved from the baseline by an absolute value of −2.1(%), almost a 26 percent reduction in 3 months after the procedure.

### Example 6. Clinical Trial, Weight Loss

In another example, multiple arteries/tissues were treated for managing body weight. The patient B with 3 years of obesity history had his body weight improved after the ablation procedure. The treated multiple arteries were hepatic, splenic, celiac, right renal and left renal arteries. The treatments can be found in Example 4.

Body weight: base=83 kg, 2 wks=79.3 kg, 1 mo=79.9 kg, 3 mos=79.1 kg. The initial body weight loss was seen at 2 weeks follow-up time at −4.5% from the baseline of 83 kg to 79.3 kg. The improved body weight was maintained at 3 months follow-up time.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Embodiments

The following exemplary embodiments are provided, the numbering of which is not to be construed as designating levels of importance:

Embodiment 1 provides a method for treating at least one disease (e.g., one disease, two diseases, at least two disease, three diseases, at least three diseases, four diseases, or at least four diseases) comprising treating at least two different target tissues (e.g., at least three different target tissues, or at least four different target tissues) in at least two different body lumens (e.g., at least three different body lumens, or at least four different body lumens), the method comprising:

performing a treatment procedure on a body lumen that is a first body lumen, the treatment procedure comprising
inserting the delivery catheter into the body lumen, wherein the delivery catheter comprises a catheter shaft, a balloon at a distal end of the shaft, and an inflation lumen in fluid communication with an interior of the balloon;
inflating the balloon to center the distal end of the shaft in the body lumen;
denervating or ablating target tissue of the body lumen with the delivery catheter comprising delivering an amount of energy or formulation to the target tissue effective to injure or damage the target tissue to relieve a disease symptom;
deflating the balloon; and
removing the delivery catheter from the body lumen;

performing the treatment procedure on a second body lumen different than the first body lumen.

Embodiment 2 provides the method of Embodiment 1, wherein performing the treatment procedure on the second body lumen comprises reusing the same delivery catheter in the treatment procedure on the second body lumen as used in the treatment procedure on the first body lumen.

Embodiment 3 provides the method of any one of Embodiments 1-2, wherein performing the treatment procedure on the second body lumen comprises using a different delivery catheter in treatment procedure on the second body lumen than used in the treatment procedure on the first body lumen, the different delivery catheter comprising a catheter shaft, a balloon at a distal end of the shaft, and an inflation lumen in fluid communication with an interior of the balloon.

Embodiment 4 provides the method of any one of Embodiments 1-3, wherein

the target tissue of the first body lumen and the second body lumen are different and are independently selected from target tissue of renal arteries, pulmonary arteries, vascular lumens, celiac arteries, common hepatic arteries, proper hepatic arteries, gastroduodenal arteries, right and left hepatic arteries, splenic arteries, right and left gastric arteries, suprarenal arteries, phrenic arteries, mesenteric arteries, nonvascular lumens, airways, the sinuses, the esophagus, respiratory and digestive lumens, the stomach, the duodenum, the jejunum, the prostate, the urethra, the ureter, and urological lumens; or

wherein the first body lumen and the second body lumen belong to different classes of body lumens chosen from renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, and urological lumens; or

a combination thereof.

Embodiment 5 provides the method of any one of Embodiments 1-4, wherein the disease is chosen from hypertension, pulmonary hypertension, diabetes, obesity, metabolic syndrome, heart failure, myocardial infarction, atherosclerosis, coronary artery disease (CAD), peripheral vascular disease (PAD), end-stage renal disease, a digestive disease, nonalcoholic fatty liver disease (NAFLD), nonalcoholic steatohepatitis (NASH), urological disease, cancers, tumors, pain, rheumatoid arthritis (RA), asthma, chronic obstructive pulmonary disease (COPD), and a combination thereof.

Embodiment 6 provides the method of any one of Embodiments 1-5, wherein the relieving the disease symptom comprises relieving a symptom of hypertension, diabetes, obesity, coronary disease, peripheral disease, nonalcoholic fatty liver disease (NAFLD), nonalcoholic steatohepatitis (NASH), cancer, arthritis, or a combination thereof.

Embodiment 7 provides the method of any one of Embodiments 1-6, wherein the relieving the disease symptom comprises reducing blood pressure, reducing blood glucose level and A1C, reducing body weight, reducing restenosis, reducing liver fat, and reducing pain, or a combination thereof.

Embodiment 8 provides the method of any one of Embodiments 1-7, wherein the delivery catheter further comprises a guide wire lumen extending at least through the distal end of the shaft, wherein the method further comprises advancing the delivery catheter over the guidewire.

Embodiment 9 provides the method of any one of Embodiments 1-8, wherein the delivery catheter further comprises a marker band on the balloon or adjacent to the balloon, wherein the method further comprises monitoring a position of the marker band under fluoroscopy.

Embodiment 10 provides the method of any one of Embodiments 1-9, wherein the delivery catheter comprises a chemical infusion delivery catheter.

Embodiment 11 provides the method of any one of Embodiments 1-10, wherein the delivery catheter comprises a combination chemical infusion delivery catheter and energy-delivery catheter.

Embodiment 12 provides the method of any one of Embodiments 1-11, wherein the delivery catheter comprises an energy-delivery catheter.

Embodiment 13 provides the method of any one of Embodiments 1-12, wherein denervating or ablating target tissue of the body lumen with the delivery catheter comprises delivering an amount of energy to the target tissue from the delivery catheter using radiofrequency, cryoablation, microwave, laser, ultrasound, high-intensity focused ultrasound, vapor condensation of at least some of the formulation to a liquid, or a combination thereof.

Embodiment 14 provides the method of any one of Embodiments 1-13, wherein the disease comprises

renal hypertension and diabetes, or

renal hypertension and obesity, or

diabetes and obesity, or

a combination thereof.

Embodiment 15 provides the method of any one of Embodiments 1-13, wherein the disease comprises renal hypertension and diabetes.

Embodiment 16 provides the method of any one of Embodiments 1-13, wherein the disease comprises renal hypertension and obesity.

Embodiment 17 provides the method of any one of Embodiments 1-13, wherein the disease comprises diabetes and obesity.

Embodiment 18 provides the method of any one of Embodiments 1-17, wherein the first or second body lumen comprises the splenic artery.

Embodiment 19 provides the method of any one of Embodiments 1-18, wherein the first or second body lumen comprises a renal artery (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof).

Embodiment 20 provides the method of any one of Embodiments 1-19, wherein the first or second body lumen comprises the hepatic artery, the hepatic artery branch, the right hepatic artery, the left hepatic artery, the common hepatic artery, the proper hepatic artery, the hepatic celiac artery, or a combination thereof.

Embodiment 21 provides the method of any one of Embodiments 1-20, wherein first body or second body lumen comprises the a renal artery (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof), wherein the method results in renal norepinephrine reduction of at least 40%.

Embodiment 22 provides a method for treating a disease, the method comprising:

inserting a delivery catheter into a body lumen, wherein the delivery catheter comprises a catheter shaft, at least one spray hole, and at least one marker band;

spraying a formulation through the at least one spray hole, wherein the amount of the formulation delivered is effective to injure or damage target tissue to relieve a disease symptom;

optionally removing the formulation from the tissue; and

withdrawing the delivery catheter from the body lumen.

Embodiment 23 provides the method of Embodiment 22, wherein the delivery catheter comprises at least one centering balloon, wherein the method further comprises inflating the centering balloon to center the delivery catheter shaft in body lumen; and deflating the centering balloon after the spraying.

Embodiment 24 provides the method of any one of Embodiments 22-23, wherein the delivery catheter comprises at least one injection needle and an infusion lumen in fluid communication with the at least one injection needle, wherein the method further comprises

deploying the at least one needle into a wall of the body lumen, outside the wall, or inside the wall; and

infusing a formulation through the infusion lumen and through the at least one needle, wherein the amount of the formulation delivered is effective to injure or damage target tissue to relieve a disease symptom; and

retracting the at least one needle into the delivery catheter after the infusion.

Embodiment 25 provides the method of any one of Embodiments 1-24, wherein the at least one needle comprises at least two needles and the delivery catheter further comprises a needle lumen, a needle control mechanism, and a flushing lumen, wherein the at least two needles are deployed from the needle lumen and retracted back into the needle lumen by the needle control mechanism, and a distal end of the flushing lumen is in fluid communication with the needle lumen, the method further comprising flushing the needles with a flushing fluid via the flushing lumen.

Embodiment 26 provides the method of any one of Embodiments 1-25, wherein the at least two needles comprise three needles and the three needles are arranged a uniform distance apart around a circumference of the catheter shaft and are located distal to the balloon.

Embodiment 27 provides a method for treating a disease, the method comprising:

inserting a centering balloon delivery catheter into a body lumen, wherein the balloon delivery catheter comprises at least one centering balloon and a catheter shaft, at least one injection needle, and at least one marker band;

inflating the centering balloon to center the delivery catheter shaft in body lumen;

deploying the at least one needle into a wall, outside the wall, or inside the wall of the body lumen;

infusing a formulation through the at least one needle, wherein the amount of the formulation delivered is effective to injure or damage target tissue to relieve a disease symptom;

optionally removing the formulation from the tissue;

retracting the needles into the delivery catheter and deflating the centering balloon; and

withdrawing the delivery catheter from the body lumen.

Embodiment 28 provides the method according to any one of Embodiments 1-27, wherein the disease is chosen from hypertension, pulmonary hypertension, diabetes, obesity, metabolic syndrome, heart failure, myocardial infarction, atherosclerosis, coronary artery disease (CAD), peripheral vascular disease (PAD), end-stage renal disease, a digestive disease, nonalcoholic fatty liver disease (NAFLD), nonalcoholic steatohepatitis (NASH), urological disease, cancers, tumors, pain, rheumatoid arthritis (RA), asthma, chronic obstructive pulmonary disease (COPD), and a combination thereof.

Embodiment 29 provides the method according to Embodiment 28, wherein cancers are chosen from adrenal, bladder, cervical, colon, esophageal, gallbladder, kidney, liver, lung, ovarian, pancreatic, prostatic, rectal, stomach, duodenum, jejunum, uterine, and a combination thereof.

Embodiment 30 provides the method according to any one of Embodiments 1-29, wherein the target tissue is tissue of renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, urological lumens, or a combination thereof.

Embodiment 31 provides the method according to any one of Embodiments 1-30, wherein the formulation comprises or consists essentially of ethanol.

Embodiment 32 provides the method according to any one of Embodiments 1-31, wherein the formulation consists of ethanol.

Embodiment 33 provides the method according to any one of Embodiments 1-32, wherein the formulation comprises a gas, vapor, liquid, solution, emulsion, suspensions of one or more ingredients, or a combination thereof.

Embodiment 34 provides the method according to any one of Embodiments 1-33, wherein the formulation comprises a vapor of one or more ingredients and heat is generated by condensation of the vapor into liquid.

Embodiment 35 provides the method according to any one of Embodiments 1-34, wherein the formulation comprises a liquid or solution and heat is transferred from the formulation to the target tissue.

Embodiment 36 provides the method according to any one of Embodiments 1-35, wherein the formulation comprises an emulsion or a suspension and heat is transferred from the formulation to the target tissue.

Embodiment 37 provides the method according to any one of Embodiments 1-36, wherein the temperature of the formulation ranges from 40 to 140° C.

Embodiment 38 provides the method according to any one of Embodiments 1-37, wherein the temperature of the formulation ranges from 0 to 140° C.

Embodiment 39 provides the method according to any one of Embodiments 1-38, wherein the temperature of the formulation ranges from −40 to 0° C.

Embodiment 40 provides the method according to any one of Embodiments 1-39, wherein the temperature of the formulation is at room temperature.

Embodiment 41 provides the method according to any one of Embodiments 1-40, wherein the temperature of the target tissue is lower than the temperature of the formulation.

Embodiment 42 provides the method according to any one of Embodiments 1-41, wherein the temperature of the target tissue is higher than the temperature of the formulation.

Embodiment 43 provides the method according to any one of Embodiments 1-42, wherein the pressure of the formulation infusion ranges from 0.1 to 14 atm.

Embodiment 44 provides the method according to any one of Embodiments 1-43, wherein the temperature of the target tissue ranges from −40 to 100° C.

Embodiment 45 provides the method according to any one of Embodiments 1-44, wherein the temperature of the target tissue ranges from −40 to 0° C.

Embodiment 46 provides the method according to any one of Embodiments 1-45, wherein the temperature of the target tissue equals that of body temperature.

Embodiment 47 provides the method according to any one of Embodiments 1-46, wherein the pressure of the formulation ranges from about 2 to 200 psi at a temperature ranging from about −40 to 150° C.

Embodiment 48 provides the method according to any one of Embodiments 1-47, wherein an amount of the formulation ranges from 0.2 microliters to 200 milliliters.

Embodiment 49 provides the method according to any one of Embodiments 1-48, wherein the method comprises inflating the delivery catheter in the body lumen for an inflation period of about 2 seconds to about 60 minutes.

Embodiment 50 provides the method according to any one of Embodiments 1-49, wherein the method delivers an amount of heat or energy to target tissue from about 2 cal/g to about 150 cal/g.

Embodiment 51 provides the method according to any one of Embodiments 1-50, wherein the delivery catheter is a needle or needle-based delivery catheter, single balloon delivery catheter, double balloon delivery catheter, energy delivery catheter, infusion catheter, balloon infusion catheter, balloon catheter, dumbbell balloon infusion catheters, or a combination thereof.

Embodiment 52 provides the method according to any one of Embodiments 1-51, wherein the balloon inflation pressure ranges from 0.1 to 14 atm.

Embodiment 53 provides the method according to any one of Embodiments 1-52, wherein the formulation comprises one or more ingredients chosen from water, saline, hypertonic saline, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate, lipiodol, urea, and derivatives and combinations thereof.

Embodiment 54 provides the method according to any one of Embodiments 1-53, wherein the formulation comprises gases or vapors of oxygen, nitrogen, helium, argon, air, carbon dioxide, nitric oxide, water, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate, or combinations thereof.

Embodiment 55 provides the method according to any one of Embodiments 1-54, wherein the formulation comprises a therapeutic agent for nerve denervation, wherein the therapeutic agent comprises sodium channel blockers, tetrodotoxins, saxitoxins, decarbamoyl saxitoxins, vanilloids, neosaxitoxins, lidocaines, conotoxins, cardiac glycosides, digoxins, glutamates, staurosporines, amlodipines, verapamils, cymarins, digitoxins, proscillaridins, quabains, veratridines, domoic acids, oleandrins, carbamazepines, aflatoxins, guanethidines, guanethidine sulfates, or a combination thereof.

Embodiment 56 provides the method according to any one of Embodiments 1-55, wherein the formulation comprises a contrast agent for imaging nerve denervation, wherein the contrast agent comprises iodine, ethyl iodide, sodium iodide, lipiodol, nonoxynol iodine, iobitridol, iohexol, iomeprol, iopamidol, iopentol, iopromide, ioversol, ioxilan, iotrolan, iodixanol, ioxaglate, derivatives thereof, or a combination thereof.

Embodiment 57 provides the method according to any one of Embodiments 1-56, wherein the formulation comprises an azeotrope.

Embodiment 58 provides the method according to Embodiment 57, wherein the azeotrope comprises ethanol/water, propanol/water, isopropanol/water, butanol/water, acetic acid/water, lactic acid/water, ethyl lactate/water, ethyl lactate/ethanol, lactic acid/ethanol/water, ethyl lactate/water/ethanol, ethyl acetate/ethanol, ethyl nitrate/ethanol, isopropyl acetate/ethanol, or a combination thereof.

Embodiment 59 provides the method according to any one of Embodiments 1-58, wherein the formulation comprises ethanol, ethanol/water, ethanol/water/oxygen, ethanol/water/air, ethanol/water/contrast agent, ethanol/water/surfactant, ethanol/water/contrast agent/surfactant, propanol/water, isopropanol/water, butanol/water, acetic acid/water, or a combination thereof.

Embodiment 60 provides a centering balloon catheter for delivery of materials to a target location in the body lumen of a patient, the centering balloon catheter comprising:

a proximal end;

a distal end;

a wire lumen;

a balloon inflation lumen;

a formulation infusion lumen and/or a vacuum lumen;

an expandable balloon section;

at least one injection needle;

at least one marker band adjacent to the centering balloon; and

at least one needle-exit opening adjacent to the marker band for deployment of the needle.

Embodiment 61 provides the centering balloon catheter of Embodiment 60, comprising three injection needles.

Embodiment 62 provides the centering balloon catheter of any one of Embodiments 60-61, wherein deployment of the needle through the needle-exit opening allows the formulation to penetrate into the wall of the body lumen at a pressure higher than that of the body lumen.

Embodiment 63 provides the centering balloon catheter of any one of Embodiments 60-62, wherein the target tissue is target tissue of renal arteries, renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, urological lumens, or a combination thereof.

Embodiment 64 provides the centering balloon catheter of any one of Embodiments 60-63, wherein the pressure of the formulation infusion ranges from 0.1 to 14 atm, and the balloon inflation pressure ranges from 0.1 to 14 atm.

Embodiment 65 provides a needle-based balloon delivery catheter for delivery of materials to a target tissue in the body lumen of a patient, the delivery catheter comprising:

a catheter shaft with a proximal and distal end;

at least one marker band located near the distal end of the shaft;

at least one needle situated in a needle lumen, wherein the needle lumen is open to the outside of the catheter shaft via at least one needle exit hole;

a flushing port at the proximal end of the shaft in fluid communication with a flushing lumen, the flushing lumen being in fluid communication with a distal end of the needle lumen, wherein the flushing port is in fluid communication with the needle exit hole through the flushing lumen;

a guide wire lumen which extends through at least the distal end of the shaft;

at least one balloon adjacent to the distal end of the catheter;

an inflation lumen;

an inflation port in fluid communication with the inflation lumen and in fluid communication with an interior of the balloon, wherein the balloon is inflatable via the inflation port through the inflation lumen and approximately centers the distal end of the catheter shaft in a body lumen;

an ablation or denervation port at the proximal end of the shaft, wherein the ablation or denervation port is in fluid communication with the at least one needle for supplying ablation energy or formulation to the at least one needle; and

a needle movement controller in electrical or mechanical communication with the at least one needle, wherein the needle movement controller deploys the at least one needle into the body lumen, into a wall of the body lumen, or outside of the body lumen.

Embodiment 66 provides the delivery catheter of Embodiment 65, comprising three injection needles.

Embodiment 67 provides the delivery catheter of any one of Embodiments 65-66, wherein deployment of the needle through the needle-exit opening allows the formulation to penetrate into the wall of the body lumen at a pressure higher than that of the body lumen.

Embodiment 68 provides the delivery catheter of any one of Embodiments 65-67, wherein the target tissue is target tissue of renal arteries (e.g., left main renal artery, right main renal artery, a renal artery branch, a left renal artery branch, a right renal artery branch, a distal end of the left main renal artery, a distal end of the right main renal artery, a distal end of a left main renal artery branch, a distal end of a right main renal artery branch, or a combination thereof), renal veins, gastric arteries, gastric veins, hepatic arteries, hepatic veins, pulmonary arteries, pulmonary veins, celiac arteries, celiac veins, gastroduodenal artery, gastroduodenal vein, splenic arteries, splenic veins, suprarenal arteries, suprarenal veins, phrenic arteries, phrenic veins, mesenteric arteries, mesenteric veins, airways, esophagus, stomach, duodenum, jejunum, urological lumens, or a combination thereof.

Embodiment 69 provides the delivery catheter of any one of Embodiments 65-68, wherein the pressure of the formulation infusion ranges from 0.1 to 14 atm, and the balloon inflation pressure ranges from 0.1 to 14 atm.

Embodiment 70 provides a delivery catheter comprising:

a shaft with a proximal and distal end;

one or more needles for infusion treatment arranged near the distal end of the shaft;

an inflatable balloon arranged near the distal end of the shaft such that when the delivery catheter is placed in a lumen and the balloon is inflated, the distal end of the catheter shaft is centered in the lumen; and

a marker band at the distal end of the shaft.

Embodiment 71 provides a delivery catheter comprising:

a shaft with a proximal and distal end;

one or more needles for infusion treatment arranged near the distal end of the shaft; and

a steering mechanism associated with the shaft such that the distal end of the shaft is steerable in a direction away from the longitudinal axis of the shaft.

Embodiment 72 provides the delivery catheter of any one of Embodiments 70-71, wherein the one or more needles comprises one needle.

Embodiment 73 provides the delivery catheter of any one of Embodiments 70-71, wherein the one or more needles comprises more than one needle.

Embodiment 74 provides the delivery catheter of Embodiment 73, wherein the one or more needles comprises a tip-to-tip needle span diameter of 5 mm to 80 mm, as measured when the one or more needles are fully advanced from the delivery catheter.

Embodiment 75 provides the delivery catheter of Embodiments 73-74, wherein the delivery catheter comprises a fixed tip-to-tip needle span diameter as measured when the one or more needles are fully advanced from the delivery catheter.

Embodiment 76 provides the delivery catheter of any one of Embodiments 73-75, wherein the delivery catheter comprises an adjustable tip-to-tip needle span diameter as measured when the one or more needles are fully advanced from the delivery catheter.

Embodiment 77 provides the delivery catheter of any one of Embodiments 70-71 and 73-76, wherein the one or more needles comprise three needles.

Embodiment 78 provides the delivery catheter of any one of Embodiments 70-77, wherein the one or more needles comprise a shape-memory material.

Embodiment 79 provides the delivery catheter of any one of Embodiments 70-78, wherein the one or more needles comprises nitinol.

Embodiment 80 provides the delivery catheter of any one of Embodiments 70-79, wherein the one or more needles comprise one or more radiopaque materials.

Embodiment 81 provides the delivery catheter of any one of Embodiments 70-80, wherein the one or more needles comprise a thin film comprising the one or more radiopaque materials.

Embodiment 82 provides the delivery catheter of any one of Embodiments 70-81, whereon the one or more needles comprise one or more radiopaque materials comprising tungsten (W), gold (Au), tantalum (Ta), platinum (Pt), iridium (Ir), a compound thereof, or a combination thereof.

Embodiment 83 provides the delivery catheter of any one of Embodiments 70-82, wherein the shaft comprises a wire lumen that is an over-the-wire (OTW) shaft.

Embodiment 84 provides the delivery catheter of any one of Embodiments 70-83, wherein the shaft comprises a wire lumen that is a rapid exchange (RX) shaft.

Embodiment 85 provides the delivery catheter of any one of Embodiments 70-84, wherein the shaft comprises one or more needle-exit openings at the distal end of the shaft.

Embodiment 86 provides the delivery catheter of any one of Embodiments 71-85, wherein a needle-exit opening is located at a side of the shaft in a direction of movement of the steering mechanism.

Embodiment 87 provides the delivery catheter of any one of Embodiments 70-86, wherein the shaft comprises at least one spray hole.

Embodiment 88 provides the delivery catheter of any one of Embodiments 85-87, wherein the shaft comprises a flushing lumen that connects to the needle-exit opening.

Embodiment 89 provides the delivery catheter of Embodiment 88, wherein the flushing lumen is for carrying a flushing fluid, wherein the flushing fluid is saline, heparinized saline, a therapeutic medicine, or a combination thereof.

Embodiment 90 provides the delivery catheter of any one of Embodiments 87-89, further comprising a sleeve over at least some of the spray holes.

Embodiment 91 provides the delivery catheter of any one of Embodiments 70-90, wherein the shaft comprises at least one vacuum hole.

Embodiment 92 provides the delivery catheter of any one of Embodiments 70-91, wherein the delivery catheter comprises an ablation or denervation port at the proximal end of the shaft, wherein the ablation or denervation port is in fluid communication with the at least one needle for supplying ablation energy or formulation to the one or more needles.

Embodiment 64 provides the method, centering balloon catheter, or delivery catheter of any one or any combination of Embodiments 1-63 optionally configured such that all elements or options recited are available to use or select from.

What is claimed is:

1. A method for treating at least one disease comprising treating at least two different target tissues in at least two different body lumens, the method comprising:
performing a treatment procedure on a body lumen that is a first body lumen, the treatment procedure comprising
inserting the delivery catheter into the body lumen, wherein the delivery catheter comprises a catheter shaft, a balloon at a distal end of the shaft, and an inflation lumen in fluid communication with an interior of the balloon;
inflating the balloon to center the distal end of the shaft in the body lumen;
denervating or ablating target tissue of the body lumen with the delivery catheter comprising delivering an amount of energy or formulation to the target tissue effective to injure or damage the target tissue to relieve a disease symptom;
deflating the balloon; and
removing the delivery catheter from the body lumen;
performing the treatment procedure on a second body lumen different than the first body lumen.

2. The method of claim 1, wherein the first body lumen and the second body lumen belong to different classes of body lumens chosen from renal arteries, renal veins, gastric arteries, hepatic arteries, pulmonary arteries, celiac arteries, gastroduodenal artery, splenic arteries, suprarenal arteries, phrenic arteries, mesenteric arteries, airways, esophagus, stomach, duodenum, jejunum, and urological lumens.

3. The method of claim 1, wherein the at least one disease comprises:
at least two diseases; or
at least three diseases; or
at least four diseases.

4. The method of claim 1, wherein the disease is chosen from hypertension, pulmonary hypertension, diabetes, obesity, metabolic syndrome, heart failure, myocardial infarction, atherosclerosis, coronary artery disease (CAD), peripheral vascular disease (PAD), end-stage renal disease, digestive disease, nonalcoholic fatty liver disease (NAFLD), nonalcoholic steatohepatitis (NASH), urological disease, cancers, tumors, pain, rheumatoid arthritis (RA), asthma, chronic obstructive pulmonary disease (COPD), and a combination thereof.

5. The method of claim 1, wherein the at least one disease comprises at least two diseases comprising
renal hypertension and diabetes, or
renal hypertension and obesity, or
diabetes and obesity, or
a combination thereof.

6. The method of claim 1, wherein the relieving the disease symptom comprises reducing blood pressure, reducing blood glucose level and A1C, reducing body weight, reducing restenosis, reducing liver fat, and reducing pain, or a combination thereof.

7. The method of claim 1, wherein the delivery catheter comprises a chemical infusion delivery catheter.

8. The method of claim 1, wherein the delivery catheter comprises an energy-delivery catheter.

9. The method of claim 1, wherein denervating or ablating target tissue of the body lumen with the delivery catheter comprises delivering an amount of energy to the target tissue from the delivery catheter using radiofrequency, cryoablation, microwave, laser, ultrasound, high-intensity focused ultrasound, vapor condensation of at least some of the formulation to a liquid, or a combination thereof.

10. The method of claim 1, wherein the formulation comprises one or more ingredients chosen from water, saline, hypertonic saline, phenol, methanol, ethanol, absolute alcohol, isopropanol, propanol, butanol, isobutanol, ethylene glycol, glycerol, acetic acid, lactic acid, propyl iodide, isopropyl iodide, ethyl iodide, methyl acetate, ethyl acetate, ethyl nitrate, isopropyl acetate, ethyl lactate, lipiodol, urea, and derivatives and combinations thereof.

11. The method of claim 1, wherein the formulation comprises ethanol.

12. The method of claim 1, wherein the formulation comprises an azeotrope.

13. A needle-based balloon delivery catheter for delivery of materials to a target tissue in the body lumen of a patient, the delivery catheter comprising:
a catheter shaft with a proximal and distal end;
at least one marker band located near the distal end of the shaft;
at least one needle situated in a needle lumen, wherein the needle lumen is open to the outside of the catheter shaft via at least one needle exit hole;
a flushing port at the proximal end of the shaft in fluid communication with a flushing lumen, the flushing lumen being in fluid communication with a distal end of the needle lumen, wherein the flushing port is in fluid communication with the needle exit hole through the flushing lumen;
a guide wire lumen which extends through at least the distal end of the shaft;
at least one balloon adjacent to the distal end of the catheter;
an inflation lumen;
an inflation port in fluid communication with the inflation lumen and in fluid communication with an interior of the balloon, wherein the balloon is inflatable via the inflation port through the inflation lumen and approximately centers the distal end of the catheter shaft in a body lumen;
an ablation or denervation port at the proximal end of the shaft, wherein the ablation or denervation port is in fluid communication with the at least one needle for supplying ablation energy or formulation to the at least one needle; and
a needle movement controller in electrical or mechanical communication with the at least one needle, wherein the needle movement controller deploys the at least one needle into the body lumen, into a wall of the body lumen, or outside of the body lumen.

14. An infusion catheter comprising:
a shaft with a proximal and distal end; and
one or more needles for infusion treatment arranged near the distal end of the shaft;
wherein the infusion catheter comprises
an inflatable balloon arranged near the distal end of the shaft such that when the infusion catheter is placed in a lumen and the balloon is inflated, the distal end of the catheter shaft is centered in the lumen, wherein the distal end of the shaft comprises a marker band, or
a steering mechanism associated with the shaft such that the distal end of the shaft is steerable in a direction away from the longitudinal axis of the shaft, or
a combination thereof.

15. The infusion catheter of claim 14, wherein the one or more needles comprises more than one needle.

16. The infusion catheter of claim 15, wherein the needles comprises a tip-to-tip needle span diameter of 5 mm to 80 mm, as measured when the needles are fully advanced from the infusion catheter.

17. The infusion catheter of claim 14, wherein the one or more needles comprise a shape-memory material.

18. The infusion catheter of claim 14, wherein the one or more needles comprises nitinol.

19. The infusion catheter of claim 14, wherein the one or more needles comprise one or more radiopaque materials.

20. The infusion catheter of claim 14, wherein the shaft comprises
at least one spray hole, or,
a flushing lumen that connects to the needle-exit opening, or
at least one vacuum hole, or
a combination thereof.
